(19) United States
(12) Patent Application Publication
(10) Pub. No.: US 2023/0158204 A1
(43) Pub. Date: May 25, 2023

(54) ANTI-FOULING IMPLANTABLE MATERIAL AND METHOD OF MAKING

(71) Applicant: Edwards Lifesciences Corporation, Irvine, CA (US)

(72) Inventors: Ekaterina Tkatchouk, Huntington Beach, CA (US); Angela B. De La Fuente, San Clemente, CA (US); Jingjia Han, Irvine, CA (US); Bin Tian, Irvine, CA (US); Liqiong Gui, Irvine, CA (US)

(21) Appl. No.: 18/157,371

(22) Filed: Jan. 20, 2023

Related U.S. Application Data
(63) Continuation of application No. PCT/US2021/042345, filed on Jul. 20, 2021.
(60) Provisional application No. 63/055,293, filed on Jul. 22, 2020.

(51) Int. Cl. A61L 27/34; C09D 5/16; A61L 27/50

(52) U.S. Cl. CPC A61L 27/34; A61L 27/50; C09D 5/1637

(57) ABSTRACT

An anti-fouling implantable material and a method of making the anti-fouling implantable material are disclosed. The anti-fouling implantable material includes a polymeric reinforcement layer, an intermediate layer comprising a protective polymer membrane, and an outer layer comprising an ionic polymer. The anti-fouling implantable material may have chemical and/or physical properties compatible with body tissue properties. The anti-fouling implantable material may be used for implantable medical devices, such as prosthetic heart valves and vascular grafts, among others.

### CROSS-REFERENCE TO RELATED APPLICATIONS

This application is a continuation of PCT application No. PCT/US2021/042345, filed Jul. 20, 2021, which claims the benefit of U.S. Pat. Application No. 63/055,293, filed Jul. 22, 2020, which is incorporated by reference herein in its entirety.

### FIELD

The present disclosure concerns an anti-fouling implantable material and a method for making the anti-fouling implantable material.

### BACKGROUND

One of the major aspects of implantable prostheses (such as prosthetic heart valve, vascular graft) constructed with animal-derived pericardial tissue is the challenge associated with time-consuming tissue-processing techniques. Additionally, animal-derived tissue can have one or more of highly variable thickness, softness, and mechanical properties. This variability can lead to extremely low yields, and/or additional costly and lengthy quality checkpoints during manufacturing.

The pericardium is a mechanically strong, double-layered membrane which surrounds the heart. Pericardial tissue consists of very compact layers of fibrous collagen and thin elastin fibers that are all interconnected by chemical bonds between each other. The fibrotic nature of the pericardium allows for its incredible strength, and the collagen’s soft and hydrophilic structure creates an environment suitable for cell proliferation.

The disadvantages of pericardial tissue have spurred the search for a material incorporating its positive properties, while overcoming the negatives, for example, a synthetic material. A concern with synthetic leaflet materials (SLMs) is the host immune response leading to fibrosis of the SLM, thereby significantly limiting leaflet performance and lifespan. The surface physicochemical properties of the SLM play an important role in modulating the fibrotic response.

### SUMMARY

This disclosure concerns an anti-fouling implantable material, as well as implantable medical devices including the anti-fouling implantable material. A method of making the anti-fouling implantable material also is disclosed.

In some examples, the anti-fouling implantable material includes (i) a reinforcement layer comprising a plurality of polymeric filaments comprising a filament polymer, the reinforcement layer having a first surface and an opposing surface; (ii) an intermediate layer comprising a protection membrane attached to at least a portion of the first surface, the protection membrane comprising a protective polymer; and (iii) an outer layer comprising an ionic polymer grafted onto an exposed surface of the intermediate layer.

In any of the foregoing or following examples, the polymeric filaments may (i) be randomly oriented, (ii) be aligned unidirectionally, (iii) form an interwoven mesh, (iv) form an intra-lamellar mesh, (v) form a knitted material, or (vi) be twisted into a yarn, which then is arranged as described in any one of (i)-(v). In any of the foregoing or following examples, the filament polymer may comprise a natural or synthetic polymer. In one example, the filament polymer is biostable. In another example, the filament polymer is biodegradable. In any of the foregoing or following examples, the polymeric filaments may comprise a core and a shell surrounding the core, wherein the core comprises the filament polymer and the shell comprises a shell polymer. The shell polymer may be a biodegradable polymer or a biostable polymer. In any of the foregoing examples, the polymeric filaments may have an average diameter within a range of from 0.001 µm to 2000 µm. In some examples, the polymeric filaments are nanofilaments or microfilaments having an average diameter within a range of from 0.001 µm to 50 µm. In any of the foregoing or following examples, the reinforcement layer may have a thickness within a range of 25-500 µm, a burst strength within a range of 50-800 N, a tensile strength within a range of 50-800 N, or any combination thereof.

The intermediate layer comprises a protection membrane attached to at least a portion of the first surface of the reinforcement layer, the protection membrane comprising a protective polymer. The protective polymer may be a biostable polymer or a biodegradable polymer. In any of the foregoing or following examples, the intermediate layer may further include a second protection membrane attached to at least a portion of the opposing surface of the reinforcement layer, the second protection membrane comprising a protective polymer. In any of the foregoing or following examples, the intermediate layer may have (i) an average thickness within a range of 0.1-100 µm, (ii) a durometer Shore hardness within a range of 10 A-80 A, (iii), a flexural modulus within a range of ₁-₅₀ N/mm², (iv) a dry ultimate tensile strength within a range of 10-60 N/mm², (v) a wet ultimate tensile strength within a range of 5-40 N/mm², or (vi) any combination of (i), (ii), (iii), (iv), and (v).

The outer layer comprises an ionic polymer grafted onto an exposed surface of the intermediate layer. The ionic polymer may be an anionic polymer, a cationic polymer, or a zwitterionic polymer. In some examples, the ionic polymer is a polyampholyte or polybetaine. In any of the foregoing or following examples, the outer layer may have an average thickness within a range of 0.001-25 µm.

Examples of a method for making an anti-fouling implantable material include forming an intermediate layer comprising a protection membrane on at least a portion of a first surface of the reinforcement layer, the reinforcement layer comprising a plurality of polymeric filaments comprising a filament polymer, and the protection membrane comprising a protective polymer; and forming an outer layer by grafting an ionic polymer onto an exposed surface of the intermediate layer. In some examples, the intermediate layer further comprises a second protection membrane on at least a portion of an opposing surface of the reinforcement layer, the second protection membrane comprising a protective polymer.

In any of the foregoing or following examples, the method further may include forming the reinforcement layer. In any of the foregoing or following examples, the method also may include forming the plurality of polymeric filaments. In some examples, forming the polymeric filaments includes forming a core comprising the filament polymer and a shell surrounding the core, the shell comprising a shell polymer.

In any of the foregoing or following examples, forming the intermediate layer comprising the protection membrane may include attaching the protection membrane to at least a portion of the first surface of the reinforcement layer. In some examples, forming the intermediate layer comprising the protection membrane further comprises forming the protection membrane. The protection membrane may be formed and then attached to the reinforcement layer. Alternatively, the protection membrane may be formed in situ on the surface of the reinforcement layer.

In any of the foregoing or following examples, grafting the ionic polymer onto the exposed surface of the intermediate layer may comprise coating the exposed surface with a solution comprising the ionic polymer to form an ionic polymer-coated material; and drying the ionic polymer-coated material to provide the anti-fouling implantable material. In some examples, the ionic polymer is a zwitterionic polymer.

The foregoing and other objects, features, and advantages will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing one example of an implantable material.

FIGS. 2A-2F show several reinforcement layer arrangements of polymeric filaments: FIG. 2A is a schematic diagram showing randomly oriented filaments; FIG. 2B is a schematic diagram showing unidirectionally aligned filaments; FIG. 2C is a schematic diagram showing an intra-lamellar mesh comprising the filaments; FIG. 2D is a schematic diagram showing an interwoven mesh comprising the filaments; FIG. 2E is a microscope image of a knitted material comprising the filaments; FIG. 2F is a scanning electron microscope image of a material knitted from yarn fibers comprising a plurality of polymeric filaments.

FIG. 3 is a schematic diagram showing one example of a polymeric filament comprising a core fiber and a shell surrounding the core fiber.

FIGS. 4A and 4B are microscope images of a reinforcement layer comprising woven fibers having a poly(lactic acid) core and a polycarbonate-urethane (PCU) shell (FIG. 4A), and a synthetic leaflet material comprising the reinforcement layer sandwiched between two thermoplastic PCU protective membranes (FIG. 4B).

FIGS. 5A and 5B are microscope images of a reinforcement layer knitted from yarn comprising filaments having a poly(ethylene terephthalate) (PET) core fiber and a hydrolyzed PET shell (FIG. 5A), and a synthetic leaflet material comprising the reinforcement layer with a thermoplastic PCU protective membrane formed by dip-coating the reinforcement layer (FIG. 5B).

FIG. 6 is a microscope image of a reinforcement layer knitted from a yarn comprising PET filaments.

FIGS. 7A and 7B are graphs comparing the burst strength (FIG. 7A) and tensile strength (FIG. 7B) of PET cloth (SLM-₁) and fixed pericardium tissue (average tissue).

FIGS. 8A and 8B are microscope images of a knitted reinforcement layer comprising filaments having a poly(ethylene terephthalate) (PET) core and a hydrolyzed PET shell (upper half) and a thermoplastic PCU protective membrane covering part of the reinforcement layer (lower half) (FIG. 8A, 30x magnification); FIG. 8B shows the PCU-covered reinforcement layer of FIG. 8A (right half), and the reinforcement layer covered with two layers of the PCU protective membrane (left half) (100x magnification).

FIGS. 9A-9C are scanning electron microscope (SEM) images of full coverage of a knitted reinforcement layer with a 127 µm thermoplastic PCU film, the knitted reinforcement layer comprising filaments having a PET core fiber and a hydrolyzed PET shell (FIGS. 9A, 103x magnification), uncoated reinforcement layer (left side) partially coated with a 127 µm thermoplastic PCU film (right side) (FIG. 9B, 100x magnification), and partial coverage of the reinforcement layer with a 127 µm thermoplastic PCU film, with defects in the coverage (FIG. 9C, 75x magnification).

FIG. 10 is X-ray images of calcified and clean explanted PET-PCU synthetic leaflet material samples after an in vivo calcification rabbit study.

FIGS. 11A-11C are energy-dispersive X-ray spectroscopy (EDS)/SEM layered (FIG. 11A), carbon (FIG. 11B), and oxygen (FIG. 11C) images of a synthetic leaflet material (SLM) comprising PET core-shell filaments and a thermoplastic PCU protection membrane

FIGS. 12A-12D are EDS/SEM layered (FIG. 12A), carbon (FIG. 12B), oxygen (FIG. 12C), and phosphorus (FIG. 12D) images of an SLM comprising PET core-shell filaments and a thermoplastic PCU protection membrane coated with 2-methacryloyloxyethyl phosphorylcholine.

FIG. 13 shows FTIR spectra of a SLM comprising PET core-shell filaments and a thermoplastic PCU protection membrane with and without a coating comprising 2-methacryloyloxyethyl phosphorylcholine.

FIG. 14 is a perspective view of an exemplary transcatheter prosthetic heart valve, according to one example.

FIG. 15 is a perspective view of an exemplary surgical prosthetic heart valve, according to one example.

### DETAILED DESCRIPTION

This disclosure concerns examples of an anti-fouling implantable material and a method for making the anti-fouling implantable material. In some examples, the anti-fouling implantable material includes a reinforcement layer, an intermediate layer comprising a protection membrane, and an outer layer comprising an ionic polymer grafted onto the intermediate layer. Some examples of the disclosed anti-fouling implantable materials are useful in implantable medical devices such as prosthetic heart valves and/or vascular grafts. The anti-fouling implantable material may exhibit reduced fibrotic, homolytic, and/or immunogenic responses compared to similar implantable materials that do not include the outer layer.

### I. Definitions and Abbreviations

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing examples from discussed prior art, the example numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley’s Condensed Chemical Dictionary, published by John Wiley & Sons, Inc., 2016 (ISBN 978-1-118-13515-0).

In order to facilitate review of the various examples of the disclosure, the following explanations of specific terms are provided:

Biodegradable: As used herein, the term biodegradable means capable of being decomposed or broken down within the body.

Biostable: As used herein, the term biostable means remaining chemically stable within the body.

Copolymer: A polymer formed from polymerization of two or more different monomers.

Elastomer: As defined by IUPAC, an elastomer is a polymer that displays rubberlike elasticity. A polymer that can be stretched with application of force and returns to its original shape when released.

Filament: A threadlike structure, a fiber. Unless specified otherwise, the term “microfilament” as used herein refers to a filament having an average diameter of from 1 µm to 100 µm. The term “nanofilament” refers to a filament having an average diameter of less than 1 µm.

Hydrogel: A cross-linked three-dimensional network of polymeric chains that are capable of absorbing and retaining molecules (e.g., water, polar solvents, non-polar solvents, drugs in liquid form, or the like) in their three-dimensional networks. Hydrogel-forming polymeric chains comprise one or more hydrophilic functional groups in their polymeric structures, such as amino (NH₂), hydroxyl (OH), amide (—CONH—, —CONH₂), sulfate (—SO₃H), or any combination thereof, and can be natural-, or synthetic-polymeric-based networks.

Hydrolyze: Decompose by reaction with water. Hydrolysis of large molecules, e.g., polymers, can be partial or complete. For example, cellulose can be hydrolyzed to form smaller polysaccharides and/or glucose.

Membrane: A thin, pliable sheet of synthetic or natural material. As used here, the term protection membrane refers to a membrane that inhibits biodegradation of an underlying material for at least a period of time.

Mesh: A knitted, woven, or knotted material of open texture, made from a network of filaments or yarn.

Monomer: A molecule or compound, usually containing carbon, that can react and combine to form polymers.

MPC: 2-methacryloyloxyethyl phosphorylcholine

PCU: polycarbonate-urethane or polycarbonate polyurethane

PET: poly(ethylene terephthalate)

PGS: poly(glycerol sebacate)

PGSU: poly(glycerol sebacate)/thermoplastic polyurethane

Polyampholyte: A polymer having anionic and cationic groups on different monomers within the polymer.

Polybetaine: A polymer comprising betaine monomers. A betaine monomer includes both anionic and cationic groups.

Polymer: A molecule of repeating structural units (e.g., monomers) formed via a chemical reaction, e.g, polymerization.

Protective polymer: As used herein, the term protective polymer refers to a polymer that inhibits biodegradation of an underlying material for at least a period of time.

SLM: Synthetic leaflet material

Subject: An animal (human or non-human) subjected to a treatment, observation or experiment.

Thermoplastic: Refers to a plastic that is capable of being heated and softened multiple times.

TPU: Thermoplastic polyurethane

UPy: Ureidopyrimidinone

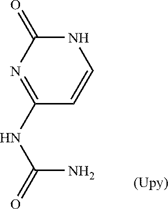

Yarn: A continuous, often plied strand composed of a plurality of fibers or filaments.

Zwitterion: A molecule or ion having separate positively and negatively charged groups.

### II. Anti-Fouling Implantable Material

The term “fouling” refers to non-specific protein absorption on a surface of at least a portion of an implanted material. These proteins can result in cellular responses, including cell attachment, wound healing, inflammation, encapsulation, or any combination of these responses. An “anti-fouling” material reduces or eliminates at least some non-specific protein absorption. Some examples of anti-fouling materials exhibit selective protein absorption, for example, proteins that promote cell attachment without triggering at least one of inflammation, immune response, encapsulation, or fibroblast proliferation. Some examples of anti-fouling materials exhibit at least one of reduced pannus formation and reduced calcification. In some examples, the anti-fouling materials exhibit no calcification, as evidenced by X-ray or inductive plasma mass spectroscopy analyses, for at least 90 days following intramuscular implantation.

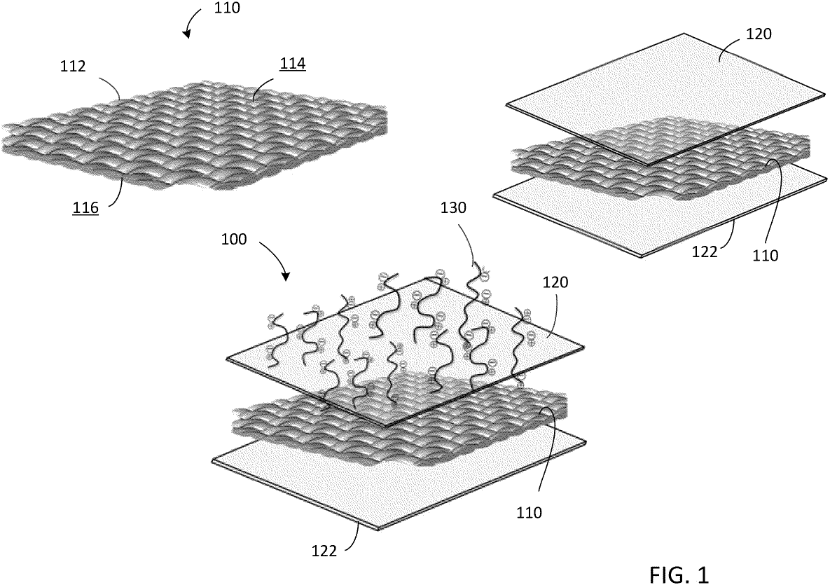

Examples of an anti-fouling implantable material are disclosed. In some examples, as shown in FIG. 1, the anti-fouling implantable material 100 comprises a reinforcement layer 110 comprising a plurality of polymeric filaments 112, the reinforcement layer having a first surface 114 and an opposing surface 116. In the example of FIG. 1, the polymeric filaments 112 are woven to form a cloth. An intermediate layer comprising a protection membrane 120 is disposed over or attached to at least a portion of the first surface 114. In certain examples, the intermediate layer further comprises a second protection membrane 122 disposed over or attached to at least a portion of the opposing surface 116. The anti-fouling implantable material 100 further comprises an outer layer comprising an ionic polymer 130 grafted onto an exposed surface of the protection membrane 120. An ionic polymer also may be grafted onto an exposed surface of the second protection membrane 122 (not shown in the view of FIG. 1).

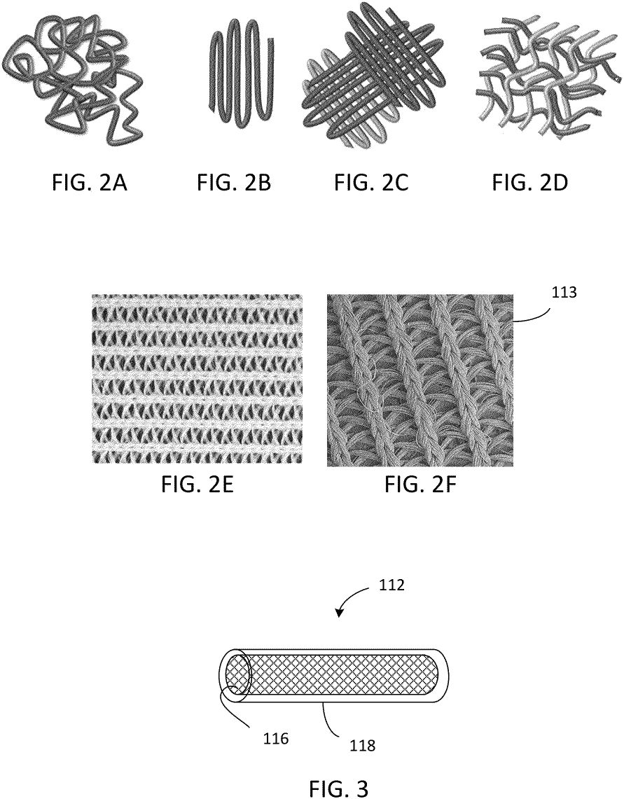

The reinforcement layer 110 comprises a plurality of polymeric filaments 112, the polymeric filaments comprising a filament polymer. The polymeric filaments 112 may be arranged in several different ways to form the reinforcement layer 110. In one arrangement, as shown in FIG. 1 and FIG. 2A, the polymeric filaments are randomly oriented to form a reinforcement layer comprising entangled filaments. In another arrangement (FIG. 2B), the polymeric filaments are aligned unidirectionally. In yet another arrangement (FIG. 2C), the polymeric filaments form an intra-lamellar mesh comprising a plurality of lamellae, wherein polymeric filaments in each lamella have a common extending direction, and polymeric filaments in adjacent lamellae are oriented in different extending directions. In still another arrangement (FIG. 2D), the polymeric filaments form an interwoven mesh comprising a first plurality of polymeric filaments having a first common extending direction interwoven with a second plurality of polymeric filaments having a second common extending direction, the second common extending direction orthogonal to the first common extending direction. In another arrangement (FIG. 2E), the polymeric filaments are knitted to form a knitted material. In some examples, the polymeric filaments are twisted into yarn fibers 113 (see FIG. 2F) comprising a plurality of polymeric filaments. The yarn fibers subsequently may be (i) randomly oriented to form a material comprising randomly oriented, entangled yarn fibers, (ii) aligned unidirectionally, (iii) woven to form an interwoven mesh, (iv) aligned to form an intra-lamellar mesh comprising a plurality of lamellae, or (v) knitted to form a knitted material (FIG. 2F).

In any of the foregoing examples, the filament polymer may comprise a biostable polymer or a biodegradable polymer. The polymer may be a synthetic polymer or a natural polymer. In some examples, the filament polymer comprises a polyurethane, a polyether ketone, a poly(ethylene terephthalate), a polycarbonate, a polyester, a polyacrylate, a polysiloxane, an aromatic polyolefin, an aliphatic polyolefin, a polyamide, a glycerol-ester polymer, a polycarboxylic acid, a polysulfone, a polysaccharide, a polyamine, a polyamino acid, a polypeptide, or any combination thereof. Suitable polyurethanes include polyester polyurethanes, polyether polyurethanes, and polycarbonate polyurethanes. The terms polyether polyurethane, polyether-urethane, and polyether-based polyurethane are used interchangeably. Similarly, the terms polycarbonate polyurethane, polycarbonate-urethane, and polycarbonate-based polyurethane are used interchangeably. Exemplary polyamides include nylons. Exemplary polycarboxylic acids include polylactic acids and poly(lactic-co-glycolic acids). Suitable polysaccharides include, but are not limited to chitin, cellulose, hyaluronate, chondroitin, and chondroitin-4-sulfate. Suitable polypeptides include, but are not limited to, silk and gelatin. In certain examples, the filament polymer comprises poly(ethylene terephthalate), poly(lactic acid), poly(lactic-co-glycolic acid), poly(glycerol sebacate), polyethylene, polypropylene, chitosan, cellulose, collagen, silk, fibrin, gelatin, and combinations thereof. In one example, the filament polymer is a biodegradable polymer, e.g., poly(lactic acid), poly(lactic-co-glycolic acid), a polysaccharide (e.g., chitosan, cellulose), a polyamino acid, a polypeptide (e.g., silk, gelatin), poly(glycerol sebacate), or a combination thereof. In another example, the filament is a biostable polymer, e.g., a polyurethane, a polyester, poly(ethylene terephthalate), a polycarbonate, a polysiloxane, an aromatic polyolefin, an aliphatic polyolefin, or a combination thereof. In an independent implementation, the filament polymer comprises a combination of a biostable polymer and a biodegradable polymer, e.g., a combination of silk and polyester.

In some examples, as shown in FIG. 3, the polymeric filament 112 comprises a core 116 comprising the filament polymer and a shell 118 surrounding the core, wherein the shell comprises a shell polymer. The shell may be a non-woven material. In one example, the shell polymer has a different chemical composition than the filament polymer. In another example, the shell polymer has the same chemical composition as the filament polymer. The shell may be mechanically or chemically attached to the core.

In any of the foregoing examples, the shell polymer may comprise a polyurethane (e.g., a polyester polyurethane, a polyether polyurethane, or a polycarbonate polyurethane), a polyether ketone, a poly(ethylene terephthalate), a polycarbonate, a polyacrylate, a polysiloxane, an aromatic polyolefin, an aliphatic polyolefin, a polyamide (e.g., a nylon), a glycerol-ester polymer, a polycarboxylic acid (e.g., polylactic acid, poly(lactic-co-glycolic acid)), a polysulfone, a polysaccharide (e.g., hyaluronic acid, chondroitin, chondroitin-4-sulfate, chitosan, cellulose, glycosaminoglycans), a polyamine, a polyamino acid, a polypeptide, or any combination thereof. In one example, the shell polymer is biostable, e.g., hydrolyzed poly(ethylene terephthalate) or a polyurethane (e.g., a polycarbonate polyurethane). In another example, the shell polymer is biodegradable, e.g., polylactic acid, poly(lactic-co-glycolic acid), a polysaccharide, a polypeptide, chitosan, cellulose, poly(glycerol sebacate), poly(xylitol sebacate), or a combination thereof. In one example, the core is a thermoplastic polyurethane and the shell is poly(glycerol sebacate). In another example, the core is poly(ethylene terephthalate) and the shell is hydrolyzed poly(ethylene terephthalate). In any of the foregoing examples, the shell may have an average thickness within a range of from 200 to 800 µm, such as 200-250 µm.

In any of the foregoing examples, the polymeric filaments may have an average diameter within a range of from 0.001 µm to 2000 µm. In some examples, the polymeric filament is a microfilament or a nanofilament. In certain examples, the polymeric filaments have an average diameter within a range of from 0.001 to 50 µm.

As shown in FIG. 1, an intermediate layer comprising a protection membrane 120 is disposed over or attached to at least a portion of the first surface 114 of the reinforcement layer 110, the protection membrane comprising a protective polymer. In some examples, the intermediate layer further comprises a second protection membrane 122 disposed over or attached to at least a portion of the opposing surface 115 of the reinforcement layer 110, the second protection membrane comprising a protective polymer. The protective polymers of the protection membrane and second protection membrane may have the same chemical composition or different chemical compositions. By “at least a portion” is meant at least 10%, at least 20%, at least 30%, at least 40%, at least 50%, at least 60%, at least 70%, at least 80%, or at least 90%, such as from 10-100%, 20-100%, 30-100%, 40-100%, 50-100%, 60-100%, 70-100%, 80-100%, 90-100%, or even 95-100% of the first surface, the opposing surface, or both the first surface and the opposing surface. In some examples, the intermediate layer comprising the protection membrane 120 is disposed over or attached to the entire first surface 114. In certain examples, the intermediate layer further comprises a second protection membrane 122, where the second protection membrane is disposed over or attached to the entire opposing surface 115. In any of the foregoing examples, the intermediate layer may have an average thickness within a range of from 10-250 µm, such as from 25-200 µm, 25-100 µm, 25-75 µm, or 25-50 µm. In some examples, the intermediate layer seals pores in the reinforcement layer and/or pores in the polymeric filaments or yarns comprising the polymeric filaments. The intermediate layer also may provide the anti-fouling implantable material with a uniform outer surface, such as a surface visibly free of irregularities or roughness when viewed with the naked eye or under low magnification (e.g., 5-10x).

In any of the foregoing examples, the protective polymer may comprise a biodegradable polymer or a biostable polymer. The polymer may be a synthetic polymer or a natural polymer. In some examples, the polymer is a hydrogel-forming natural or synthetic polymer. Suitable biostable synthetic polymers include, but are not limited to, polyethylene (PE) (including low density PE (LDPE) – molecular weight less than 50,000 g/mol, high density PE (HDPE) - molecular weight 2 □ 10⁵ to 3 □ 10⁶ g/mol, and ultrahigh molecular weight PE (UHMWPE) - molecular weight 3-7.5 □ 10⁶ g/mol), polypropylene, polytetrafluoroethylene, polyethers, polycarbonate polyurethanes, polysiloxane polyurethanes, polyether polyurethane elastomers, polyester polyurethane elastomers, silicones, polycarbonates, polysulfones, polyether ether ketones, poly(ethylene terephthalate), polyesters, and combinations thereof. Suitable biodegradable synthetic polymers include, but are not limited to, polyesters, polyacrylates, polyamides, hydrophilic polyester polyurethanes, hydrophilic polyureas, poly(amide-enamine), a polyanhydrides, poly(ester amide)s, poly(glycolide), poly(glycerol sebacate), poly(xylitol sebacate), polylactic acid, polyglycolic acid, polycaprolactone, poly(hydroxy butyrate), poly(ℇ-caprolactone), poly(ethylene glycol) diacrylate (PEGDA), poly(₂-hydroxyethyl methacrylate) (poly(HEMA)), ureidopyrimidinone-based polymers, poly(vinyl alcohol)-hyaluronic acid, hyaluronate amines, and combinations thereof. Suitable hydrogel-forming polymers include, but are not limited to, proteins (e.g., collagen, gelatin), polysaccharides (e.g., chitosan, cellulose, starch, alginate, agarose), hydrophilic polyurethanes, poly(ethylene oxide) (PEO), polyacrylamide (PAAm), polyethylene glycols (PEG), polyacrylates, polypeptides, poly(glycerol sebacate), poly(xylitol sebacate), and combinations thereof. In some examples, the protective polymer comprises a thermoplastic polyurethane, poly(glycerol sebacate), or a combination thereof. In certain examples, the thermoplastic polyurethane comprises a polycarbonate polyurethane or a polyetherpolyurethane. In an independent example, the protective polymer comprises poly(ethylene glycol) diacrylate. In another independent example, the protective polymer comprises poly(₂-hydroxyethyl methacrylate). In still another independent example, the protective polymer comprises a ureidopyrimidinone-based polymer.

In any of the foregoing examples, the anti-fouling implantable material 100 further may comprise an outer layer comprising an ionic polymer 130 grafted onto an exposed surface of the intermediate layer comprising the protection membrane 120 (FIG. 1). An outer layer comprising an ionic polymer also may be grafted onto an exposed surface of the second protection membrane 122 (not shown in the view of FIG. 1). The ionic polymer grafted onto the exposed surface of the second protection membrane 122 may be the same as or different than the ionic polymer 130 grafted onto the exposed surface of the protection membrane 120. In any of the foregoing examples, the outer layer may have an average thickness within a range of from 0.001 µm to 25 µm. In some examples, the outer layer may have a polymer graft density from 0.1-2.5 chains/nm².

In any of the foregoing examples, the ionic polymer may be an anionic polymer, a cationic polymer, or a zwitterionic polymer. In any of the foregoing examples, the ionic polymer may have a chain length of from 5 to 500 ionic units. In some examples, the ionic polymer is a zwitterionic polymer. The zwitterionic polymer may be a polyampholyte or a polybetaine. In some examples, the zwitterionic polymer comprises a poly(phosphocholine), a poly(sulfobetaine), a poly(carboxybetaine), a zwitterionic polysaccharide, diethyl ethanolamine quaternized with ₂-acrylamide-₂-methylpropane sulfonic acid and acrylic acid, or any combination thereof. In certain examples, the zwitterionic polymers include, but are not limited to, polymers comprising ₂-methacryloyloxyethyl phosphorylcholine (MPC) moieties, sulfobetaine methacrylate (SBMA) moieties, carboxybetaine methacrylate (CBMA) moieties, or any combination thereof. In the exemplary zwitterionic monomer formulas below, m and n are integers. In some examples, m is 1 and n is 1.

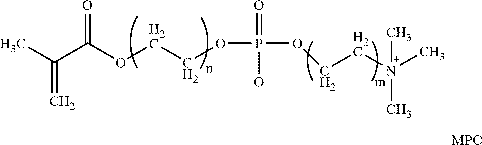

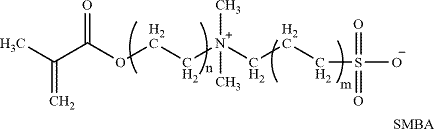

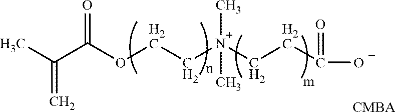

In certain examples, the ionic polymer is a copolymer, e.g., a copolymer of MPC, SMBA, or CBMA and at least one other monomer. Exemplary ionic polymers include, but are not limited to, poly(MPC-co-₂-ethylhexyl methacrylate-co-N,N-diethylaminoethyl methacrylate), poly(MPC-co-p-nitrophenyloxycarbonyl poly(ethylene glycol) methacrylate), poly(2-hydroxyethyl methacrylate)-MPC copolymers, polyvinylpyrrolidone-MPC copolymers, and combinations thereof.

In some examples, the grafted ionic polymer forms polymer brushes on the exposed surface of the protection membrane. In a polymer brush, one terminus of the polymer is attached to the surface while the other terminus is free. The polymer brush conformation or configuration may provide protein adsorption resistance and/or cell adhesion resistance when the anti-fouling implantable material is implanted into a subject.

In some examples, the ionic polymer reduces fibrotic, hemolytic, and/or immunogenic responses when the anti-fouling implantable material is implanted into a subject. The ionic polymer coating modifies the anti-fouling implantable material surface and may reduce tissue reaction by reducing fibrosis. Certain zwitterionic groups, such as phosphorylcholine, may prevent biological reactions due to their affinity to the phospholipid structure of cell membranes. Phospholipid-assembled surfaces suppress many biological responses and have excellent anti-thrombogenic responses when the polymers come in contact with platelet-rich plasma. In the absence of the ionic polymer, proteins may adsorb onto the surface within a few seconds of the material coming into contact with body fluids such as blood or plasma. For example, however, protein adsorption on MPC-containing polymers from human plasma, as determined by radioimmunoassay and an immuno-colloid labelling technique showed that the amount of adsorbed protein was quite small and decreased with an increase in the number of MPC moieties.

In one example, an anti-fouling implantable material includes a woven mesh reinforcement layer made of polymer filaments comprising a biodegradable poly(lactic acid) (PLA) core fiber and a thermoplastic polycarbonate-urethane (PCU) shell. The reinforcement layer is sandwiched between two protection membranes PCU. The outer layer comprises a zwitterionic polymer, e.g., an MPC-containing polymer, grafted onto surfaces of the PCU protection membranes.

In another example, an anti-fouling implantable material comprises a knitted cloth reinforcement layer made of poly(ethylene terephthalate) PET yarn comprising PET fibers twisted together. The PET yarn surface is hydrolyzed to provide a core-shell structure. The intermediate layer comprises a PCU protection membrane attached to exposed surfaces of the knitted PET cloth. In an independent example, the intermediate layer comprises two layers of the PCU protection membrane. The outer layer comprises a zwitterionic polymer, e.g., an MPC-containing polymer, grafted onto exposed surfaces of the intermediate layer.

In yet another example, an anti-fouling implantable material comprises a knitted cloth reinforcement layer made of PET yarn comprising PET fibers twisted together. An intermediate layer comprising an aromatic PCU protection membrane having a Shore hardness of 30 A-75 A and a thickness of 40-50 µm is applied to the entire outer surface of the reinforcement layer. The intermediate layer comprises two layers of a PCU protection membrane, each layer having a thickness of 20-25 µm). The outer layer comprises a zwitterionic polymer including 2-MPC grafted onto the intermediate layer.

In still another example, an anti-fouling implantable material comprises a knitted cloth reinforcement layer made of PET yarn comprising PET fibers twisted together. The PET yarn surface is hydrolyzed to provide a core-shell structure. The intermediate layer comprises a polyether-based hydrogel thermoplastic polyurethane protection membrane attached to an exposed surface of the reinforcement layer. The outer layer comprises a zwitterionic polymer, e.g., an MPC-containing polymer, grafted onto exposed surfaces of the intermediate layer.

In another example, an anti-fouling implantable material comprises a reinforcement layer comprising electrospun aromatic polycarbonate polyurethane filaments to provide a porous structure with both small and large pore sizes. In some examples, the pore sizes have an average diameter of from 0.1-50 µm. In certain examples, small pores may have an average diameter from 0.1-10 µm and/or large pores may have an average diameter of from 10-50 µm. The intermediate layer comprises a poly(glycerol sebacate) protective membrane having a weight-average molecular weight of from 5,000-1,000,000 g/mol, for example, 31,000 g/mol. The outer layer comprises a zwitterionic polymer including 2-MPC grafted onto the intermediate layer.

In yet another example, an anti-fouling implantable material includes a reinforcement layer comprising electrospun filaments, the filaments comprising two co-spun polymers: an aliphatic, hydrophilic polyether-based polyurethane hydrogel and a biostable aromatic polycarbonate polyurethane. The intermediate layer comprises a PEG-based hydrogel protection membrane. The outer layer comprises a zwitterionic polymer including 2-MPC grafted onto the intermediate layer.

Advantageously, some examples of the disclosed anti-fouling implantable materials have chemical and/or physical properties compatible with body tissue properties. For example, in certain examples, the anti-fouling implantable materials have properties compatible with pericardium tissue and/or vascular tissue. In any of the foregoing examples, the reinforcement layer may have a burst strength within a range of from 50-1000 N (measured per ASTM D3787-01), such as a burst strength within a range of from 500-800 N. In any of the foregoing examples, the reinforcement layer may have a tensile strength (wet or dry, measured per ASTM D412) within a range of from 20-800 N, such as within a range of from 50-300 N. In any of the foregoing examples, the intermediate layer may have (i) a durometer Shore hardness within a range of 10 A-80 A (ASTM D785), (ii) a flexural modulus within a range of from 1-50 N/mm² (ASTM D790), (iii) a dry ultimate tensile strength within a range of from 10-60 N/mm² (ASTM D412) (iv) a wet ultimate tensile strength within a range of from 5-40 N/mm² (ASTM D412), (v) a dry ultimate elongation within a range of from 25-500% (ASTM D412), (vi) a wet ultimate elongation within a range of from 25-500% (ASTM D412), or (vii) any combination thereof. In any of the foregoing examples, the anti-fouling implantable material may have a burst strength within a range of from 50-1000 N, such as within a range of from 500-800 N.

Without being bound by any theory, it is believed that materials with Shore hardnesses in the state ranges allow for natural coaptation of leaflets manufactured therefrom, although coaptation in some examples of leaflets can be improved by methods including shape setting or heat setting.

In any of the foregoing examples, the filament polymer (including the core fiber polymer and/or shell polymer), the protective polymer, or both may be biodegradable. In certain examples, a biodegradable material may allow for tissue regeneration when the anti-fouling implantable material is implanted into a body. In some examples, the filament polymer (including the core fiber polymer and/or shell polymer), the protective polymer, or both may be biostable. In any of the foregoing examples, the anti-fouling implantable material may comprise a combination of biostable and biodegradable polymers. As just one non-limiting example, the filament polymer may be biostable while the protective polymer is biodegradable.

In any of the foregoing examples, the anti-fouling implantable material can be used to form an implantable medical device or a component of an implantable medical device. In some examples, the implantable medical device comprises a prosthetic heart valve, wherein the anti-fouling implantable material can be used to form the prosthetic leaflets of the prosthetic valve or other soft components of the prosthetic valve, such as a sealing skirt or a covering for metal components of the prosthetic valve. In certain examples, the implantable medical device is a surgically implantable prosthetic heart valve for replacing any of the native heart valves (the aortic, mitral, tricuspid and pulmonary valves). In other examples, the implantable medical device is a transcatheter prosthetic heart valve for replacing any of the native heart valves. Exemplary patents and publications relating to prosthetic heart valves in which examples of the disclosed anti-fouling implantable material may be useful include US 7,993,394; US 8,252,051; US 8,454,685; US 8,568,475; US 9,393,110; US 9,636,223; US 9,662,204; US 9,974,650; US 9,974,652; US 10,195,025; US 10,226,334; US 10,363,130; US 10,413,407; US 10,426,611; US 10,433,958; US 10,433,959; US 2018/0028310; US 2019/0167422A1; and WO 2018/222799, each of which is incorporated herein by reference in its entirety for all purposes.

In other examples, the implantable medical device can comprise a docking device for receiving a prosthetic heart valve at a location within the heart, such as disclosed in U.S. Pat. Publication Nos. 2019/0000615 and 2017/0231756 and U.S. Pat. No. 10,463,479, which are incorporated herein by reference in their entireties for all purposes. The anti-fouling material disclosed herein can be incorporated in such docking devices where anti-fouling properties are desired. For example, the anti-fouling material can be used to form an inner layer and/or an outer layer of a docking device.

In other examples, the implantable medical device can comprise a valve repair device for repairing a native heart valve (any of the aortic, mitral, tricuspid or pulmonary valves). Repair device can include, for example, complete or partial annuloplasty rings; a leaflet clipping device, such as disclosed in U.S. Pat. Publication No. 2016/0331523 and U.S. Pat. No. 10,524,913; or a leaflet augmentation device, such as disclosed U.S. Pat. Publication No. 2015/0230919, the entire disclosures all of which are incorporated herein by reference for all purposes. The anti-fouling material disclosed herein can be incorporated in such valve repair devices where anti-fouling properties are desired. For example, the anti-fouling material can be used to form an outer layer or covering for a repair device, such as a tubular covering for an annuloplasty ring.

In other examples, the implantable medical device can be a cardiovascular patch or a vascular graft.

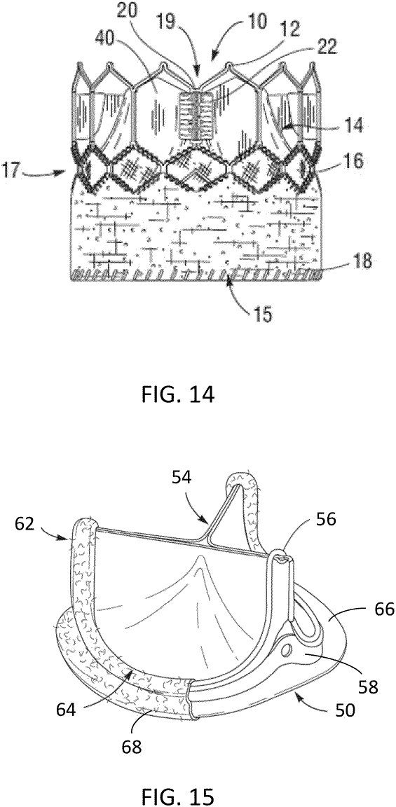

FIG. 14 shows a transcatheter prosthetic heart valve ₁₀, according to one example, configured to be implanted via catherization, as known in the art. The illustrated prosthetic valve is adapted to be implanted in the native aortic valve annulus, although other examples are adapted for replacing other native heart valves (e.g., the pulmonary, mitral, and tricuspid valves). The prosthetic valve can also be adapted to be implanted in other tubular organs or passageways in the body. The prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12 while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in other examples there can be greater or fewer number of leaflets (e.g., one or more leaflets 40). The leaflets 40 can be secured to one another at their adjacent sides to form commissures 22 of the leaflet structure 14. The lower edge of valvular structure 14 can have an undulating, curved scalloped shape and can be secured to the inner skirt 16 by sutures (not shown). The leaflets 40 can be formed of an anti-fouling implantable material as disclosed herein. In some examples, it may be desirable to form the inner skirt 16 and/or the outer skirt 18 of an anti-fouling implantable material as disclosed herein.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (e.g., stainless steel, etc.) or self-expanding materials (e.g., nickel titanium alloy (NiTi), such as nitinol), as known in the art. In some examples, when constructed of a plastically-expandable material, the frame 12 (and thus the prosthetic valve 10) can be crimped to a radially collapsed configuration on a delivery catheter and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, without limitation, stainless steel; biocompatible, high-strength alloys (e.g., cobalt-chromium or nickel-cobalt-chromium alloys); polymers; or combinations thereof. In particular examples, frame 12 is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight. Additional details regarding the prosthetic valve 10 and its various components are described in WIPO Patent Application Publication No. WO 2018/222799, which is incorporated herein by reference for all purposes.

FIG. 15 shows a perspective view of an exemplary prosthetic heart valve 50 according to one example. The prosthetic heart valve 50 can be implanted in an open-heart procedure, as known in the art. The heart valve 50 comprises a plurality of (usually three) flexible leaflets 54 supported partly by an undulating wireform 56, a support band 58, and a sewing ring 66. The wireform 56 defines a support frame for the leaflets 54. The wireform 56 may be formed from a suitably elastic metal, such as a Co—Cr—Ni alloy (e.g., Elgiloy® alloy), while the support band or stent may be metallic, plastic, or a combination of the two. The wireform 56 defines an undulating periphery of alternating commissures 62 and cusps 64 to which the leaflets 54 are secured. Each commissure 62 is located intermediate two arcuate cusps 34 that curve toward the inflow direction. The wireform 56, support band 58, and sewing ring 66 is typically covered with a polyester fabric 68 as shown to facilitate assembly and to reduce direct blood exposure after implant. The leaflets 54 can be formed of an anti-fouling implantable material as disclosed herein. In some examples, the polyester fabric 68 may be replaced with an anti-fouling implantable material as disclosed herein.

### III. Method of Making the Anti-Fouling Implantable Material

In some examples, a method of making an anti-fouling implantable material as disclosed herein forming an intermediate layer comprising a protection membrane on at least a portion of a first surface of the reinforcement layer, the reinforcement layer comprising a plurality of polymeric filaments comprising a filament polymer, and the protection membrane comprising a protective polymer. The method may further include forming an outer layer by grafting an ionic polymer onto an exposed surface of the intermediate layer. In any of the foregoing examples, the intermediate layer may further comprise grafting a second protection membrane on at least a portion of an opposing surface of the reinforcement layer, the second protection membrane comprising a protective polymer.

In any of the foregoing examples, the method may further comprise forming the reinforcement layer. In one example, forming the reinforcement layer comprises jet spinning, electro-spinning, or melt spinning the plurality of polymeric filaments to form a material comprising randomly oriented, entangled filaments. In an independent example, forming the reinforcement layer comprises aligning the plurality of polymeric filaments unidirectionally. In another independent example, forming the reinforcement layer comprises weaving the plurality of polymeric filaments to form an interwoven mesh comprising a first plurality of filaments having a first common extending direction interwoven with a second plurality of filaments having a second common extending direction, the second common extending direction orthogonal to the first common extending direction. In yet another independent example, forming the reinforcement layer comprises aligning the plurality of polymeric filaments to form an intra-lamellar mesh comprising a plurality of lamellae, wherein filaments in each lamella have a common extending direction, and filaments in adjacent lamellae are oriented in different extending directions. In yet another independent example, forming the reinforcement layer comprises knitting the plurality of polymeric filaments to form a knitted material. In still another independent example, forming the reinforcement layer comprises twisting the plurality of polymeric filaments to form yarn fibers, and subsequently (i) randomly orienting the yarn fibers for form a material comprising randomly oriented, entangled yarn fibers, (ii) aligning the yarn fibers unidirectionally, (iii) weaving the yarn fibers to form an interwoven mesh, (iv) aligning the yarn fibers to form an intra-lamellar mesh comprising a plurality of lamellae, or (v) knitting the yarn fibers to form a knitted material. In still another independent example, forming the reinforcement layer comprises printing the plurality of polymeric filaments in a pattern by three-dimensional printing. The pattern may be any desired pattern, e.g., a single layer of aligned polymeric filaments, an intra-lamellar mesh, etc.

In any of the foregoing examples, the method may further comprise forming the plurality of polymeric filaments comprising the filament polymer. Suitable methods for forming the polymeric filaments include, but are not limited to, jet spinning, electro-spinning, melt spinning, three-dimensional printing, extrusion, or a melt-blown process.

In some examples, the polymeric filaments comprise a core comprising the filament polymer and a shell surrounding the core, the shell comprising a shell polymer. In one example, the core and shell are formed in a single step by jet spinning (e.g., using a highspeed rotating nozzle), electro-spinning, co-extrusion, or three-dimensional printing. In some examples, the filament polymer and shell polymer are provided in a solution, a melt, a two-part composition (e.g., an epoxy), or a suspension. In certain examples, the core is formed as described above, and then coated with the shell polymer to form the shell. For example, the shell may be formed by dipping the core fiber in a molten shell polymer and allowing the shell polymer to cool around the core fiber. In another example, the shell is formed in a solvent-based process by dipping the core fiber in a solution comprising the polymer and allowing the solvent to evaporate, thereby depositing the shell polymer onto the core fiber. In another independent example, the core is formed as described above, and a surface of the core is then hydrolyzed to form a shell.

In any of the foregoing examples, forming the intermediate layer may further comprise forming the protection membrane. In one example, the protection membrane is formed by melting or extruding the protective polymer to form a film. In an independent example, the protective polymer may be compression molded to form a film. In another independent example, the protective polymer is dissolved in a solvent to form a solution comprising the protective polymer; a film is then formed from the solution. Depending on the protective polymer, suitable solvents may include methanol, ethanol, propanol, 2-propanol, 1-butanol, 2-butanol, t-butyl alcohol, acetone, acetonitrile, 2-butanone, chloroform, trichloromethane, dimethoxyethane, trifluoroethanol, diglyme, diethyl ether, methyl t-butyl ether, methylene chloride, ethyl acetate, ethylene glycol, glycerol, dimethylsulfoxide (DMSO), dimethylformamide (DMF), acetic acid, tetrahydrofuran (THF), 2-methyltetrahydrofuran, dimethylacetamide (DMAc or DMA), dioxane, heptane, dihydrolevoglucosenone (Cyrene™ solvent, Sigma-Aldrich), polyethylene glycol (MW 400), aqueous-based buffers (e.g., 3-(N-morpholino)propanesulfonic acid (MOPS), tris(hydroxymethyl)aminomethane (Tris), and phosphate-buffered saline (PBS) buffers), and mixtures thereof. Mixed solvents may include water and an organic solvent combined in a ratio of from 1:2 v/v to 1:20 v/v. In one example, the protective polymer comprises a thermoplastic polyurethane and the polymer is dissolved in a solution comprising dimethylacetamide, tetrahydrofuran, or a combination thereof.

In any of the foregoing examples, the intermediate layer comprising the protection membrane may be attached to or disposed over at least a portion of the first surface by any suitable method. Suitable methods include, but are not limited to, thermal attachment, mechanical attachment, ultrasonic attachment, laser attachment, chemical attachment, solvent-based attachment, and three-dimensional printing. In some examples, the intermediate layer further comprises a second protection membrane similarly attached to or disposed over at least a portion of the opposing surface of the reinforcement layer.

In some examples, the intermediate layer comprising the protection membrane is thermally attached by heat pressing the protection membrane to the reinforcement layer surface (e.g., over at least a portion of the first surface, and optionally, over at least a portion of the opposing surface), molding the protection membrane on the surface, or extruding the protective polymer onto the surface. Heat pressing is performed at a temperature and time effective to adhere the protection membrane to the reinforcement layer surface without crystallizing the filament polymer or protective polymer. In one example, a thermoplastic aromatic polyurethane protection membrane was adhered to a PET reinforcement layer at a temperature within a range of 190-200° C. and a pressure of 0.7-0.8 N/mm² for 15 seconds.

In some examples, the intermediate layer comprising the protection membrane is mechanically attached to the reinforcement layer surface. Mechanical attachment may comprise pressing the protection membrane onto the surface in the absence of added heat. Alternatively, mechanical attachment may be enhanced by changing the surface morphology of the protection membrane. Surface modification of the protection membrane can be performed by processes including, but not limited to, laser ablation, ion milling, and sputter etching.

In an independent example, the intermediate layer comprising the protection membrane is ultrasonically attached to the reinforcement layer surface. In another example, the intermediate layer comprising the protection membrane is attached to the reinforcement layer surface using a laser.

In some examples, the intermediate layer comprising the protection membrane is chemically attached to the reinforcement layer surface. In such examples, the protective polymer includes functional groups capable of reacting with functional groups on the reinforcement layer surface, for example, functional groups on the filament polymer or, in the case of a core-shell filament, the shell polymer. Chemical attachment may be performed by hydrolysis or oxidation of the reinforcement layer surface and the protective polymer, whereby chemical functional groups of the filament polymer or shell polymer react with functional groups of the protective polymer. In some examples, hydrolysis is performed using acetic acid and/or sodium hydroxide. Oxidation may be performed with hydrogen peroxide. In another example, ultraviolet, plasma, or corona-processing techniques are used to alter the surface chemistry and chemically attach the intermediate layer to the reinforcement layer surface.

In some examples, the intermediate layer comprising the protection membrane is formed in situ on the reinforcement layer surface. For example, the reinforcement layer may be coated with a solution comprising the protective polymer. Coating may be performed by any suitable method including, but not limited to dip coating, spray coating, or spin coating. The solution viscosity is adjusted so that the dissolved protective polymer moves slowly as the solvent evaporates. In some examples, the solvent is evaporated to form the protection membrane. In some examples, the protective polymer may be cured with ultraviolet radiation.

In some examples, the protection membrane is formed in situ by a reactive dip-coating process. As one non-limiting example, the reinforcement layer may be immersed consecutively in chemically reactive dipping solutions of poly(ethyleneglycol) (PEG) methyl ether acrylate (e.g., average Mn 480) followed by poly(ethyleneimine) (PEI) (e.g., 10-50% (w/v) in H₂O). The two polymers mutually react by a Michael addition reaction. Reactive dipping solutions may be prepared in different solvents at concentrations ranging from 2-70% (w/v). Suitable solvents include, for example, toluene, ethanol, and 1-heptanol.

In some examples, forming the intermediate layer comprising the protection membrane in situ comprises printing the protection membrane on the reinforcement layer surface by a three-dimensional printing process. In certain examples, forming the intermediate layer comprises forming two layers of the protection membrane comprising the protective polymer. The first layer seals pores in the reinforcement layer filaments or yarn fibers, and the second layer provides a uniformly coated surface on the anti-fouling implantable material.

In any of the foregoing examples, forming the outer layer by grafting the ionic polymer onto the exposed surface of the intermediate layer may comprise contacting the exposed surface with a solution comprising the ionic polymer to form an ionic polymer-coated material, and drying the ionic polymer-coated material. In some examples grafting the ionic polymer onto the exposed surface of the intermediate layer comprises spray coating ionic polymer solution onto the exposed surface. Spray coating may include plasma spraying or thermal spraying processes. In an independent example, the implantable material may be dipped into the ionic polymer solution, thereby dip-coating the implantable material with the ionic polymer. In another independent example, the ionic polymer may be vapor deposited onto the exposed surface of the intermediate layer by physical or chemical vapor deposition. If the intermediate layer comprises a second protection membrane, an ionic polymer also may be grafted onto an exposed surface of the second protection membrane. The ionic polymer grafted onto the second protection membrane may be the same or different than the ionic polymer on the protection membrane. In some examples, the ionic polymer is a zwitterionic polymer as previously discussed.

The ionic polymer may be chemically or mechanically grafted to the protection membrane. In some examples, the ionic polymer includes a side chain comprising a functional group that can react with functional groups on the protective polymer molecules, thereby chemically grafting, or bonding, the ionic polymer to the protection membrane. Suitable functional groups include, but are not limited to, an anionic group, a cationic group, a hydrogen-bonding group, a photoreactive group, or an alkoxysilane group For example, the ionic polymer may comprise a side chain terminating in a carboxylic acid (—COOH) group, which is capable of reacting with functional groups (e.g., carboxylic acid, hydroxyl, or amine groups, among others) on the protective polymer, thereby chemically binding the ionic polymer to the protection membrane. In some examples, a protection membrane, such as a protection membrane comprising polyurethane, is treated with dilute acid or plasma to produce additional carboxylic acid groups on the protection membrane surface for reaction with the ionic polymer. Chemically grafting the ionic polymer to the protection membrane comprises contacting the protection membrane with the ionic polymer under conditions effective to promote a chemical reaction between an ionic polymer functional group and a protective polymer functional group. Effective conditions may include a temperature and/or contact time effective to promote the chemical reaction. Contacting the protection membrane with the ionic polymer may comprise spray coating the protection membrane with a solution comprising the ionic polymer, vapor deposition of a solution comprising the ionic polymer onto the protection membrane, immersing the protection membrane in a solution comprising the ionic polymer, or any other suitable method. Following the reaction, the anti-fouling implantable material may be washed to remove any unbound ionic polymer and/or side products of the reaction.

In some examples, the ionic polymer includes a side chain that may be inserted between molecules of the protection membrane, thereby mechanically grafting the ionic polymer to the protection membrane. For instance, the ionic polymer may include a side chain comprising a hydrophobic group (e.g., an aliphatic group). The ionic polymer is mechanically grafted to the protection membrane by swelling the protection membrane to provide spaces between the protective polymer molecules. The protection membrane may be swelled by contact with, or immersion in, a suitable solvent. For example, some polyurethanes swell when contacted with ethanol. The swollen protection membrane is contacted with the ionic polymer, whereby at least some of the ionic polymer side chains insert into the spaces between the protective polymer molecules. Contacting the swollen protection membrane with the ionic polymer may comprise spray coating the protection membrane with a solution comprising the ionic polymer, vapor deposition of a solution comprising the ionic polymer onto the protection membrane, immersing the protection membrane in a solution comprising the ionic polymer, or any other suitable method. The anti-fouling implantable material is then dried. As the anti-fouling implantable material dries, the spaces between the protective polymer molecules close and trap the ionic polymer side chains, thereby mechanically grafting the ionic polymer to the protection membrane.

In any of the foregoing or following examples, the method may further include forming a plurality of leaflets from the implantable material and coupling the leaflets to a frame of a prosthetic heart valve.

In one example, the method of making an anti-fouling implantable material comprises providing a reinforcement layer comprising an interwoven mesh of polymeric filaments comprising poly(lactic acid). The reinforcement layer is dip-coated in a solution comprising polycarbonate-urethane (PCU), thereby forming a dip-coated reinforcement layer comprising an interwoven mesh of polymeric filaments comprising a poly(lactic acid) core fiber and a PCU shell. The dip-coated reinforcement layer has a first surface and an opposing surface. A protection membrane comprising PCU is thermally attached to at least a portion of the first surface. Optionally, a second protection membrane comprising PCU is thermally attached to at least a portion of the opposing surface. A zwitterionic polymer is chemically or mechanically grafted onto an exposed surface of the protection membrane or onto exposed surfaces of the protection membrane and the second protection membrane. In certain examples, the zwitterionic polymer comprises 2-methacryloyloxyethyl phosphorylcholine.

In another example, the method of making an anti-fouling implantable material comprises providing a reinforcement layer comprising a knitted material formed from a yarn comprising a plurality of polymeric filaments comprising poly(ethylene terephthalate) (PET). A surface of the yarn is hydrolyzed to form a hydrolyzed PET shell on the polymeric filaments comprising PET. A protection membrane comprising PCU or poly(glycerol sebacate) (PGS) is attached to at least a portion of the first surface of the reinforcement layer. In some examples, the protection membrane is attached by (i) thermally attaching the protection membrane to at least a portion of the first surface, (ii) dip-coating the reinforcement layer in a solution comprising PCU or PGS, or (iii) depositing the protection membrane onto at least a portion of the first surface by three-dimensional printing. In certain examples, the protection membrane is thermally attached to the portion of the first surface, and the method further comprises thermally attaching a second protection membrane to at least a portion of an opposing surface of the reinforcement layer. A zwitterionic polymer is chemically or mechanically grafted onto an exposed surface of the protection membrane or onto exposed surfaces of the protection membrane and the second protection membrane. In certain examples, the zwitterionic polymer comprises 2-methacryloyloxyethyl phosphorylcholine.

In yet another example, the method of making an anti-fouling implantable material comprises providing a knitted cloth reinforcement layer made of poly(ethylene terephthalate) PET yarn comprising PET fibers twisted together. The PET yarn surface is hydrolyzed to provide a core-shell structure. An intermediate layer comprising a PCU protection membrane is attached to exposed surfaces of the knitted PET cloth, e.g., by dip-coating the reinforcement layer in a solution comprising PCU. In an independent example, two layers of the PCU protection membrane are applied to the reinforcement layer. A zwitterionic polymer, e.g., MPC, may be grafted onto exposed surfaces of the PCU protection membrane.

In still another example, the method of making an anti-fouling implantable material comprises providing a knitted cloth reinforcement layer made of PET yarn comprising PET fibers twisted together. An intermediate layer comprising an aromatic PCU protection membrane having a Shore hardness of 30 A-75 A and a thickness of 40-50 µm is applied to the entire outer surface of the reinforcement layer. The intermediate layer comprises two layers of a PCU protection membrane, each layer having a thickness of 20-25 µm, which are thermally attached to the reinforcement layer. The temperature and time are selected to melt the PCU material and facilitate attachment. An outer layer comprising a zwitterionic polymer including poly(₂-methacryloyloxyethyl phosphorylcholine) is grafted onto the intermediate layer by dissolving the zwitterionic polymer in ethanol and immersing the implantable material in the polymer solution.

In another example, the method of making an anti-fouling implantable material comprises providing a knitted cloth reinforcement layer made of PET yarn comprising PET fibers twisted together. The PET yarn surface is hydrolyzed to provide a core-shell structure. A protection membrane comprising a polyether-based hydrogel thermoplastic polyurethane is attached to an exposed surface of the reinforcement layer. A zwitterionic polymer, e.g., MPC, may be grafted onto exposed surfaces of the protection membrane.

In still another example, the method of making an anti-fouling implantable material comprises forming a reinforcement layer by electrospinning filaments comprising an aromatic polycarbonate polyurethane. An intermediate layer comprising a poly(glycerol sebacate) (weight-average molecular weight 31,000 g/mol) protection membrane is thermally attached to an exposed surface of the reinforcement layer to encapsulate the reinforcement layer and form an implantable material. The implantable material is dip-coated in a solution comprising poly(₂-methacryloyloxyethyl phosphoryl choline) to form an outer layer comprising polymer brushes on the surface of the intermediate layer.

In yet another example, the method of making an anti-fouling implantable material comprising forming a reinforcement layer by simultaneously electrospinning two polyurethanes - an aliphatic, hydrophilic polyether-based polyurethane and a biostable aromatic polycarbonate polyurethane. An intermediate layer comprising a PEG-based hydrogel protection membrane is formed by dip-coating the reinforcement layer in a prepolymer solution comprising PEGDA (poly(ethylene glycol) diacrylate, 10 kDa) and 2-hydroxy-4′-(2-hydroxyethoxy)-2-methylpropiophenone (photoinitiator) in phosphate buffered saline (pH 7.4) and polymerizing under ultraviolet light.

### IV. Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An anti-fouling implantable material, comprising: a reinforcement layer comprising a plurality of polymeric filaments comprising a filament polymer, the reinforcement layer having a first surface and an opposing surface; an intermediate layer comprising a protection membrane attached to at least a portion of the first surface, the protection membrane comprising a protective polymer; and an outer layer comprising an ionic polymer grafted onto an exposed surface of the intermediate layer.

Example 2. The anti-fouling implantable material of any example herein, particularly example 1, wherein the polymeric filaments of the reinforcement layer: are randomly oriented to form a material comprising entangled polymer filaments; or are aligned unidirectionally; or form an interwoven mesh comprising a first plurality of polymeric filaments having a first common extending direction interwoven with a second plurality of polymeric filaments having a second common extending direction, the second common extending direction orthogonal to the first common extending direction; or form an intra-lamellar mesh comprising a plurality of lamellae, wherein polymeric filaments in each lamella have a common extending direction, and polymeric filaments in adjacent lamellae are oriented in different extending directions; or are knitted to form a knitted material; or are twisted into yarn fibers comprising a plurality of polymeric filaments, wherein the yarn fibers subsequently are (i) randomly oriented to form a material comprising randomly oriented, entangled yarn fibers, (ii) aligned unidirectionally, (iii) woven to form an interwoven mesh, (iv) aligned to form an intra-lamellar mesh comprising a plurality of lamellae, or (v) knitted to form a knitted material.

Example 3. The anti-fouling implantable material of any example herein, particularly example 1 or example 2, wherein the filament polymer comprises a polyurethane, a polyether ketone, a poly(ethylene terephthalate), a polycarbonate, a polyester, a polyacrylate, a polysiloxane, an aromatic polyolefin, an aliphatic polyolefin, a polyamide, a glycerol-ester polymer, a polycarboxylic acid, a polysulfone, a polysaccharide, a polyamine, a polyamino acid, a polypeptide, or any combination thereof.

Example 4. The anti-fouling implantable material of any example herein, particularly any one of examples 1-3, wherein the filament polymer comprises a synthetic polymer.

Example 5. The anti-fouling implantable material of any example herein, particularly any one of examples 1-4, wherein the filament polymer comprises a biostable polymer or a biodegradable polymer.

Example 6. The anti-fouling implantable material of any example herein, particularly example 5, wherein: the biostable polymer comprises a polyurethane, a polyester, poly(ethylene terephthalate), a polycarbonate, a polysiloxane, an aromatic polyolefin, or an aliphatic polyolefin; or the biodegradable polymer comprises poly(lactic acid), poly(lactic-co-glycolic acid), a polysaccharide, a polyamino acid, a polypeptide, or poly(glycerol sebacate).

Example 7. The anti-fouling implantable material of any example herein, particularly any one of examples 1-6, wherein the polymeric filaments comprise a core and a shell surrounding the core, wherein the core comprises the filament polymer and the shell comprises a shell polymer.

Example 8. The anti-fouling implantable material of any example herein, particularly example 7, wherein the shell polymer has a different chemical composition than the filament polymer.

Example 9. The anti-fouling implantable material of any example herein, particularly example 7 or example 8, wherein the shell polymer comprises a biodegradable polymer or a biostable polymer.

Example 10. The anti-fouling implantable material of any example herein, particularly example 9, wherein: the biodegradable polymer comprises poly(lactic acid), poly(lactic-co-glycolic acid), a polysaccharide, a polyamino acid, a polypeptide, or poly(glycerol sebacate); or the biostable polymer comprises hydrolyzed poly(ethylene terephthalate) or a polyurethane.

Example 11. The anti-fouling implantable material of any example herein, particularly any one of examples 1-10, wherein the polymeric filaments have an average diameter within a range of from 0.001 µm to 2000 µm.

Example 12. The anti-fouling implantable material of any example herein, particularly example 11, wherein: the polymeric filaments are nanofilaments or microfilaments having an average diameter within a range of from 0.001 µm to 50 µm; and at least some of the polymeric filaments are chemically, thermally, or mechanically fused with one another.

Example 13. The anti-fouling implantable material of any example herein, particularly any one of examples 1-12, wherein the reinforcement layer has: (i) a thickness within a range of from 25 µm to 500 µm; or (ii) a burst strength within a range of from 50-800 N; or (iii) a tensile strength within a range of from 50-800 N; or (iv) any combination of (i), (ii), and (iii).

Example 14. The anti-fouling implantable material of any example herein, particularly any one of examples 1-13, wherein the protective polymer comprises a biostable or biodegradable polymer.

Example 15. The anti-fouling implantable material of any example herein, particularly example 14, wherein the protective polymer comprises: a biostable synthetic polymer selected from polyethylene, polypropylene, polytetrafluoroethylene, a polyether, polycarbonate polyurethane, polysiloxane polyurethane, a polyether polyurethane elastomer, a polyester polyurethane elastomer, a silicone, a polycarbonate, a polysulfone, polyether ether ketone, poly(ethylene terephthalate), a polyester, or any combination thereof; or a biodegradable synthetic polymer selected from a polyester, a polyacrylate, a polyamide, a hydrophilic polyester polyurethane, a hydrophilic polyurea, poly(amide-enamine), a polyanhydride, a poly(ester amide), poly(glycolide), polylactic acid, polyglycolic acid, polycaprolactone, poly(hydroxy butyrate), poly(ε-caprolactone), poly(vinyl alcohol)-hyaluronic acid, a hyaluronate amine, a ureidopyrimidinone-based polymer, or any combination thereof; or a hydrogel-forming polymer selected from a protein, a polysaccharide, a hydrophilic polyurethane, poly(ethylene oxide), polyacrylamide, a polyethylene glycol, a polyacrylate, a polypeptide, poly(glycerol sebacate), poly(xylitol sebacate), or any combination thereof.

Example 16. The anti-fouling implantable material of any example herein, particularly any one of examples 1-15, wherein the intermediate layer further comprises a second protection membrane attached to at least a portion of the opposing surface of the reinforcement layer, the second protection membrane comprising a protective polymer, wherein the protective polymer of the second protection membrane may have the same or a different chemical composition than the protective polymer of the protection membrane attached to the first surface of the reinforcement layer.

Example 17. The anti-fouling implantable material of any example herein, particularly any one of examples 14-16, wherein the intermediate layer has: (i) an average thickness within a range of from 0.1 µm to 100 µm; or (ii) a durometer Shore hardness within a range of from 10 A to 80 A; or (iii) a flexural modulus within a range of from 1 N/mm² to 50 N/mm²; or (iv) a dry ultimate tensile strength within a range of from 10 N/mm² to 60 N/mm²; or (v) a wet ultimate tensile strength within a range of from 5 N/mm² to 40 N/mm²; or (vi) any combination of (i), (ii), (iii), (iv), and (v).

Example 18. The anti-fouling implantable material of any example herein, particularly any one of examples 1-17, wherein the ionic polymer is an anionic polymer, a cationic polymer, or a zwitterionic polymer.

Example 19. The anti-fouling implantable material of any example herein, particularly example 18, wherein the ionic polymer is a polyampholyte or polybetaine.

Example 20. The anti-fouling implantable material of any example herein, particularly example 18 or example 19, wherein the ionic polymer comprises a poly(phosphocholine), a poly(sulfobetaine), a poly(carboxybetaine), a zwitterionic polysaccharide, diethyl ethanolamine quaternized with 2-acrylamide-2-methylpropane sulfonic acid and acrylic acid, or any combination thereof.

Example 21. The anti-fouling implantable material of any example herein, particularly example 20, wherein the poly(phosphocholine) comprises 2-methacryloyloxyethyl phosphorylcholine (MPC) moieties.

Example 22. The anti-fouling implantable material of any example herein, particularly any one of examples 18-21, wherein the ionic polymer comprises: poly(MPC); or poly(MPC-co-2-ethylhexyl methacrylate-co-N,N-diethylaminoethyl methacrylate); or poly(MPC-co-p-nitrophenyloxycarbonyl poly(ethylene glycol) methacrylate); or a poly(2-hydroxyethyl methacrylate)-MPC copolymer; or a polyvinylpyrrolidone-MPC copolymer; or any combination thereof.

Example 23. The anti-fouling implantable material of any example herein, particularly any one of examples 1-22, wherein the outer layer has an average thickness within a range of from 0.001 µm to 25 µm.

Example 24. The anti-fouling implantable material of any example herein, particularly any one of examples 1-23, wherein the filament polymer comprises poly(lactic acid) and the intermediate layer comprises polycarbonate-urethane.

Example 25. The anti-fouling implantable material of any example herein, particularly example 24, wherein the polymeric filaments of the reinforcement layer form an interwoven mesh.

Example 26. The anti-fouling implantable material of any example herein, particularly any one of examples 1-23, wherein the filament polymer comprises poly(ethylene terephthalate) and the intermediate layer comprises polycarbonate-urethane.

Example 27. The anti-fouling implantable material of any example herein, particularly example 26, wherein the polymeric fibers are knitted to form a knitted material.

Example 28. The anti-fouling implantable material of any example herein, particularly example 26 or example 27, wherein the polymeric filaments comprise a core and a shell surrounding the core, wherein the core comprises the filament polymer and the shell comprises a shell polymer comprising hydrolyzed poly(ethylene terephthalate).

Example 29. The anti-fouling implantable material of any example herein, particularly any one of examples 1-23, wherein the filament polymer comprises poly(ethylene terephthalate) and the intermediate layer comprises a polyether-based hydrogel thermoplastic polyurethane.

Example 30. The anti-fouling implantable material of any example herein, particularly any one of examples 1-23, wherein the filament polymer comprises polycarbonate-polyurethane and the intermediate layer comprises poly(glycerol sebacate).

Example 31. The anti-fouling implantable material of any example herein, particularly example 30, wherein the intermediate layer further comprises a thermoplastic polyurethane.

Example 32. The anti-fouling implantable material of any example herein, particularly any one of examples 1-23, wherein the filament polymer comprises an aliphatic polyether-based polyurethane hydrogel and an aromatic polycarbonate polyurethane, and the intermediate layer comprises poly(ethylene glycol) diacrylate.

Example 33. The anti-fouling implantable material of any example herein, particularly any one of examples 1-23, wherein the filament polymer comprises poly(ethylene terephthalate) and the intermediate layer comprises poly(2-hydroxyethyl methacrylate).

Example 34. The anti-fouling implantable material of any example herein, particularly example 33, wherein the polymeric fibers are knitted to form a knitted material.

Example 35. The anti-fouling implantable material of any example herein, particularly any one of examples 1-23, wherein the filament polymer comprises silk.

Example 36. The anti-fouling implantable material of any example herein, particularly example 36, wherein the polymeric filaments comprise a core and a shell surrounding the core, wherein the core comprises the silk, and the shell comprises a shell polymer comprising an aromatic polycarbonate polyurethane or an aliphatic polyether polyurethane.

Example 37. The anti-fouling implantable material of any example herein, particularly any one of examples 1-23, wherein the filament polymer comprises silk and a polyester, and the intermediate layer comprises a ureidopyrimidinone-based polymer.

Example 38. The anti-fouling implantable material of any example herein, particularly example 37, wherein the ureidopyrimidinone-based polymer comprises

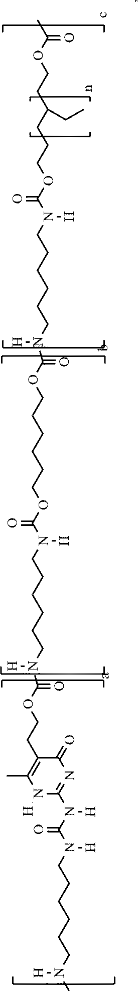

wherein a, b, and c, independently are integers greater than or equal to 1.

Example 39. The anti-fouling implantable material of any example herein, particularly example 37 or example 38, wherein the polymeric fibers are knitted to form a knitted material.

Example 40. The anti-fouling implantable material of any example herein, particularly any one of examples 1-23, wherein the filament polymer comprises gelatin, and the intermediate layer comprises a polycarbonate polyurethane and a polyether polyurethane.

Example 41. The anti-fouling implantable material of any example herein, particularly example 40, wherein the gelatin is crosslinked.

Example 42. The anti-fouling implantable material of any example herein, particularly example 40 or example 41, wherein the reinforcement layer and intermediate layer have an average combined thickness of 0.2 mm to 0.6 mm.

Example 43. The anti-fouling implantable material of any example herein, particularly any one of examples 1-23, wherein the filament polymer comprises a polycarbonate polyurethane and a polyether polyurethane, and the intermediate layer comprises poly(glycerol sebacate) and a thermoplastic polyurethane.

Example 44. The anti-fouling implantable material of any example herein, particularly example 43, wherein the reinforcement layer has an average pore size of 0.1 µm to 45 µm.

Example 45. The anti-fouling implantable material of any example herein, particularly example 43 or example 44, wherein the reinforcement layer and intermediate layer have an average combined thickness of 0.2 mm to 0.6 mm.

Example 46. The anti-fouling implantable material of any example herein, particularly any one of examples 24-46, wherein the ionic polymer comprises 2-methacryloyloxyethyl phosphorylcholine.

Example 47. An implantable medical device comprising an anti-fouling implantable material of any example herein, particularly any one of examples 1-46.

Example 48. The implantable medical device of any example, herein, particularly example 47, wherein the implantable medical device comprises a prosthetic heart valve, a vascular graft, an annuloplasty ring, a cardiovascular patch, or a coaptation clip.

Example 49. The implantable medical device of any example, herein, particularly example 47, wherein the implantable medical device comprises a prosthetic heart valve comprising a plurality of leaflets, a sealing skirt, a covering for a metal component, or any combination thereof, formed from the anti-fouling implantable material.

Example 50. A prosthetic heart valve comprising an anti-fouling implantable material, the anti-fouling material comprising: a reinforcement layer comprising a plurality of polymeric filaments comprising a filament polymer, the reinforcement layer having a first surface and an opposing surface; an intermediate layer comprising a protection membrane attached to at least a portion of the first surface, the protection membrane comprising a protective polymer; and an outer layer comprising an ionic polymer grafted onto an exposed surface of the intermediate layer.

Example 51. The prosthetic heart valve of any example herein, particularly example 50, wherein the prosthetic heart vale comprises a plurality of leaflets, a sealing skirt, a covering for a metal component, or any combination thereof, formed from the anti-fouling implantable material.

Example 52. A valve repair device comprising an anti-fouling implantable material, the anti-fouling material comprising: a reinforcement layer comprising a plurality of polymeric filaments comprising a filament polymer, the reinforcement layer having a first surface and an opposing surface; an intermediate layer comprising a protection membrane attached to at least a portion of the first surface, the protection membrane comprising a protective polymer; and an outer layer comprising an ionic polymer grafted onto an exposed surface of the intermediate layer.

Example 53. The valve repair device of any example herein, particularly example 52, wherein the valve repair device comprises an annuloplasty ring, a leaflet clipping device, or a leaflet augmentation device.

Example 54. The valve repair device of any example herein, particularly example 52 or example 53, wherein the valve repair device comprises an outer layer or covering comprising the anti-fouling implantable material.

Example 55. A cardiovascular patch or a vascular graft comprising an anti-fouling implantable material, the anti-fouling material comprising: a reinforcement layer comprising a plurality of polymeric filaments comprising a filament polymer, the reinforcement layer having a first surface and an opposing surface; an intermediate layer comprising a protection membrane attached to at least a portion of the first surface, the protection membrane comprising a protective polymer; and an outer layer comprising an ionic polymer grafted onto an exposed surface of the intermediate layer.

Example 56. A docking device for receiving a prosthetic heart valve at a location within the heart, the docking device comprising an anti-fouling implantable material, the anti-fouling material comprising: a reinforcement layer comprising a plurality of polymeric filaments comprising a filament polymer, the reinforcement layer having a first surface and an opposing surface; an intermediate layer comprising a protection membrane attached to at least a portion of the first surface, the protection membrane comprising a protective polymer; and an outer layer comprising an ionic polymer grafted onto an exposed surface of the intermediate layer.

Example 57. A method for making an anti-fouling implantable material, comprising: forming an intermediate layer comprising a protection membrane on at least a portion of a first surface of a reinforcement layer, the reinforcement layer comprising a plurality of polymeric filaments comprising a filament polymer, and the protection membrane comprising a protective polymer; and forming an outer layer by grafting an ionic polymer onto an exposed surface of the intermediate layer.

Example 58. The method of any example herein, particularly example 57, wherein the intermediate layer further comprises a second protection membrane on at least a portion of an opposing surface of the reinforcement layer, the second protection membrane comprising a protective polymer.

Example 59. The method of any example herein, particularly example 57 or example 58, further comprising forming the reinforcement layer by: jet spinning, electro-spinning, or melt spinning the plurality of polymeric filaments to form a material comprising randomly oriented, entangled filaments; or aligning the plurality of polymeric filaments unidirectionally; or weaving the plurality of polymeric filaments to form an interwoven mesh comprising a first plurality of filaments having a first common extending direction interwoven with a second plurality of filaments having a second common extending direction, the second common extending direction orthogonal to the first common extending direction; or aligning the plurality of polymeric filaments to form an intra-lamellar mesh comprising a plurality of lamellae, wherein filaments in each lamella have a common extending direction, and filaments in adjacent lamellae are oriented in different extending directions; or knitting the plurality of polymeric filaments to form a knitted material; or twisting the plurality of polymeric filaments to form yarn fibers, and subsequently (i) randomly orienting the yarn fibers for form a material comprising randomly oriented, entangled yarn fibers, (ii) aligning the yarn fibers unidirectionally, (iii) weaving the yarn fibers to form an interwoven mesh, (iv) aligning the yarn fibers to form an intra-lamellar mesh comprising a plurality of lamellae, or (v) knitting the yarn fibers to form a knitted material; or printing the plurality of polymeric filaments in a pattern by three-dimensional printing.

Example 60. The method of any example herein, particularly any one of examples 57-59, further comprising forming the plurality of polymeric filaments comprising the filament polymer by jet spinning, electro-spinning, melt spinning, three-dimensional printing, extrusion, or a melt-blown process.

Example 61. The method of any example herein, particularly any one of examples 57-60, wherein the polymeric filaments comprise a core comprising the filament polymer and a shell surrounding the core, the shell comprising a shell polymer, the method further comprising: forming the core and shell in a single step by jet spinning, electro-spinning, co-extrusion or three-dimensional printing; or forming the core and then coating the core with the shell polymer to form the shell; or forming the core, and hydrolyzing a surface of the core to form a shell.

Example 62. The method of any example herein, particularly any one of examples 57-61, further comprising forming the protection membrane by: melting or extruding the protective polymer to form a film; or dissolving the protective polymer in a solvent to form a solution comprising the protective polymer, and forming a film from the solution; or compression molding the protective polymer to form a film.

Example 63. The method of any example herein, particularly any one of examples 57-62, wherein forming the intermediate layer comprising the protection membrane on at least a portion of the first surface of the reinforcement layer further comprises: thermally attaching the protection membrane to at least a portion of the first surface; or mechanically attaching the protection membrane to at least a portion of the first surface; or ultrasonically attaching the protection membrane to at least a portion of the first surface; or attaching the protection membrane to at least a portion of the first surface using a laser; or chemically attaching the protection membrane to at least a portion of the first surface by hydrolysis or oxidation of the reinforcement layer and protective polymer, whereby chemical functional groups of the filament polymer or shell polymer react with functional groups of the protective polymer; or coating the reinforcement layer with a solution comprising the protective polymer and a solvent, and removing the solvent to form the protection membrane; or forming the protection membrane from a solution comprising the protective polymer by a reactive dip-coating process; or coating the reinforcement layer with a solution comprising the protective polymer and curing the protective polymer by ultraviolet irradiation; or printing the protection membrane onto at least a portion of the first surface by a three-dimensional printing process.

Example 64. The method of any example herein, particularly any one of examples 57-63, wherein grafting the ionic polymer onto the exposed surface of the intermediate layer comprises: coating the exposed surface with a solution comprising the ionic polymer to form an ionic polymer-coated material; and drying the ionic polymer-coated material.

Example 65. The method of any example herein, particularly any one of examples 57-64, wherein the ionic polymer is a zwitterionic polymer.

Example 66. The method of any example herein, particularly any one of examples 57-65, wherein the ionic polymer comprises a poly(phosphocholine), a poly(sulfobetaine), a poly(carboxybetaine), a zwitterionic polysaccharide, diethyl ethanolamine quaternized with 2-acrylamide-2-methylpropane sulfonic acid and acrylic acid, or any combination thereof.

Example 67. The method of any example herein, particularly example 66, wherein the poly(phosphocholine) comprises 2-methacryloyloxyethyl phosphorylcholine (MPC) moieties.

Example 68. The method of any example herein, particularly example 66 or example 67 wherein the ionic polymer comprises: poly(MPC-co-2-ethylhexyl methacrylate-co-N,N-diethylaminoethyl methacrylate); or poly(MPC-co-p-nitrophenyloxycarbonyl poly(ethylene glycol) methacrylate); or a poly(2-hydroxyethyl methacrylate)-MPC copolymer; or a polyvinylpyrrolidone-MPC copolymer; or any combination thereof.

Example 69. The method of any example herein, particularly any one of examples 57-60 or 62-68, wherein forming the reinforcement layer comprises melt-spinning poly(lactic acid) to form the plurality of polymeric fibers, and weaving the plurality of polymeric filaments to form an interwoven mesh.

Examples 70. The method of any example herein, particularly example 69, wherein forming the intermediate layer comprising the protection membrane on at least a portion of the first surface of the reinforcement layer further comprises coating the reinforcement layer with a solution comprising polycarbonate-urethane and a solvent, and removing the solvent to form the protection membrane.

Example 71. The method of any example herein, particularly any one of examples 57-68, wherein forming the reinforcement layer comprises melt-spinning poly(ethylene terephthalate) (PET) to form the plurality of polymeric fibers, twisting the plurality of polymeric fibers together to form yarn fibers, knitting the yarn fibers to form a knitted material, and hydrolyzing a surface of the polymeric fibers to form a shell comprising hydrolyzed PET on a core comprising PET.

Example 72. The method of any example herein, particularly example 71, wherein forming the intermediate layer comprising the protection membrane on at least a portion of the first surface of the reinforcement layer further comprises thermally attaching the protection membrane to at least a portion of the first surface, wherein the protection membrane comprises an aromatic polycarbonate-urethane, an aliphatic polycarbonate-urethane, or a combination thereof.

Example 73. The method of any example herein, particularly example 72, wherein thermally attaching comprises pressing the protection membrane to the first surface of the reinforcement layer at a temperature of 180° C. to 200° C. and a pressure of 0.7-0.8 N/mm² for a time of 10 seconds to 20 seconds.

Example 74. The method of any example herein, particularly example 72 or example 73, wherein the protection membrane has a thickness of 25 µm to 130 µm.

Example 75. The method of any example herein, particularly example 71, wherein forming the intermediate layer comprising the protection membrane on at least a portion of the first surface of the reinforcement layer further comprises thermally attaching the protection membrane to at least a portion of the first surface, wherein the protection membrane comprises a polyether-based hydrogel thermoplastic polyurethane.

Example 76. The method of any example herein, particularly example 75, wherein thermally attaching comprises pressing the protection membrane to the first surface of the reinforcement layer at a temperature of 190° C. to 200° C. and a pressure of 0.5-0.7 N/mm² for a time of 10 seconds to 20 seconds.

Example 77. The method of any example herein, particularly example 71, wherein forming the intermediate layer comprising the protection membrane on at least a portion of the first surface of the reinforcement layer further comprises coating the reinforcement layer with a solution comprising the protective polymer and a solvent, and removing the solvent to form the protection membrane, wherein the protective polymer comprises poly(2-hydroxyethyl methacrylate), and coating comprises spray coating.

Example 78. The method of any example herein, particularly any one of examples 57-60 or 62-68, wherein forming the reinforcement layer comprises electrospinning an aromatic polycarbonate polyurethane to form the plurality of polymeric fibers.

Example 79. The method of any example herein, particularly example 78, wherein forming the intermediate layer comprising the protection membrane on at least a portion of the first surface of the reinforcement layer further comprises chemically attaching the protection membrane by dissolving poly(glycerol sebacate) and a thermoplastic polyurethane in a solvent to form a solution, applying the solution to the first surface of the reinforcement layer, and removing the solvent.

Example 80. The method of any example herein, particularly any one of examples 57-60 or 62-68, wherein forming the reinforcement layer comprises simultaneously electrospinning an aliphatic, hydrophilic polyether-based polyurethane and an aromatic polycarbonate polyurethane.

Example 81. The method of any example herein, particularly example 80, wherein forming the intermediate layer comprising the protection membrane on at least a portion of the first surface of the reinforcement layer further comprises coating the reinforcement layer with a solution comprising the protective polymer, and curing the protective polymer by ultraviolet irradiation, wherein the protective polymer comprises poly(ethylene glycol) diacrylate.

Example 82. The method of any example herein, particularly any one of examples 57-60 or 62-68, wherein forming the reinforcement layer comprises knitting yarn fibers comprising silk to form a knitted material.

Example 83. The method of any example herein, particularly example 82, further comprising forming the protection membrane by compression molding the protective polymer to form a film, wherein the protective polymer comprises an aromatic polycarbonate polyurethane or an aliphatic polyether polyurethane.

Example 84. The method of any example herein, particularly any one of examples 57-60 or 62-68, wherein forming the reinforcement layer comprises knitting yarn fibers comprising silk and polyester to form a knitted material.

Example 85. The method of any example herein, particularly example 84, wherein forming the intermediate layer comprising the protection membrane on at least a portion of the first surface of the reinforcement layer further comprises coating the reinforcement layer with a solution comprising the protective polymer and a solvent, and removing the solvent, wherein the protective polymer comprises a ureidopyrimidinone polymer.

Example 86. The method of any example herein, particularly example 85, wherein the ureidopyrimidinone polymer comprises

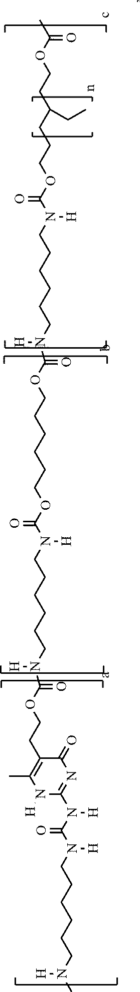

wherein a, b, and c independently are integers greater than or equal to 1.

Example 87. The method of any example herein, particularly any one of examples 84-86, wherein the reinforcement layer and the intermediate layer have a combined average thickness of 0.2 mm to 0.6 mm.

Example 88. The method of any example herein, particularly any one of examples 57-60 or 62-68, wherein forming the reinforcement layer comprises electrospinning gelatin to form the plurality of polymeric fibers.

Example 89. The method of any example herein, particularly example 88, further comprising crosslinking the gelatin.

Example 90. The method of any example herein, particularly example 88 or example 89, wherein forming the intermediate layer comprising the protection membrane on at least a portion of the first surface of the reinforcement layer further comprises coating the reinforcement layer with a solution comprising the protective polymer and a solvent, and removing the solvent, wherein the protective polymer comprises a polycarbonate polyurethane and polyether polyurethane.

Example 91. The method of any example herein, particularly any one of examples 57-60 or 62-68, wherein forming the reinforcement layer comprises electrospinning a polycarbonate polyurethane and a polyether polyurethane to form the plurality of polymeric fibers.

Example 92. The method of any example herein, particularly example 91, wherein forming the intermediate layer comprising the protection membrane on at least a portion of the first surface of the reinforcement layer further comprises coating the reinforcement layer with a solution comprising the protective polymer and a solvent, and removing the solvent, wherein the protective polymer comprises poly(glycerol sebacate) and a thermoplastic polyurethane.

Example 93. The method of any example herein, particularly example 91 or example 92, wherein the reinforcement layer and the intermediate layer have a combined average thickness of 0.2 mm to 0.6 mm.

Example 94. The method of any example herein, particularly any one of examples 57-93, wherein grafting the ionic polymer onto the exposed surface of the intermediate layer comprises: coating the exposed surface with a solution comprising poly(2-methacryloyloxyethyl phosphorylcholine) to form an ionic polymer-coated material; and drying the ionic polymer-coated material.

Example 95. The method of any example herein, particularly any one of examples 57-94, further comprising forming a plurality of leaflets from the anti-fouling implantable material and coupling the leaflets to a frame of a prosthetic heart valve.

### V. Experimental Examples

### Example 1

### Biodegradable Pla-PCU Synthetic Leaflet Material

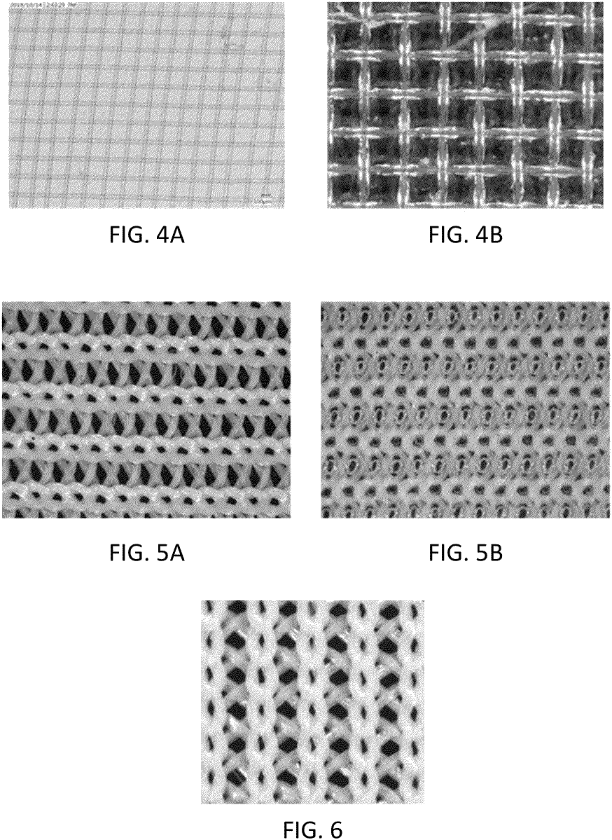

A biodegradable poly(lactic acid) (PLA) fiber was created by a melt-spinning process. The obtained PLA fiber was woven into a mesh. The mesh was dip-coated in a solution of polycarbonate-urethane (PCU) to provide a reinforcement layer having a core-shell structure. The dried reinforcement layer was sandwiched between two thin layers of PCU by heat pressing to form a synthetic leaflet material (SLM). FIGS. 4A and 4B are images of the reinforcement layer and SLM, respectively.

### Example 2

### Biostable Pet-PCU Synthetic Leaflet Materials

A biostable poly(ethylene terephthalate) fiber was created by melt-spinning and twisted together to form a yarn. The yarn was knitted into a PET cloth (FIG. 5A). The PET cloth was hydrolyzed to chemically modify the PET fiber surface and provide a reinforcement layer having a core-shell technology. Hydrolysis was formed by submerging the PET cloth in a 2.5 M NaOH solution at 50° C. for 360 minutes. The hydrolyzed cloth subsequently was submerged in 1 N acetic acid to replace sodium ions with protons. Hydrolysis was confirmed by Fourier transform infrared spectroscopy-attenuated total reflection (FTIR-ATR). Without wishing to be bound by a particular theory of operation, surface hydrolysis improves attachment of the protection membrane to the PET cloth. The reinforcement layer was dried at 50° C. overnight before applying the protection membrane. The reinforcement layer was dip-coated in a solution of PCU to form the SLM (FIG. 5B).

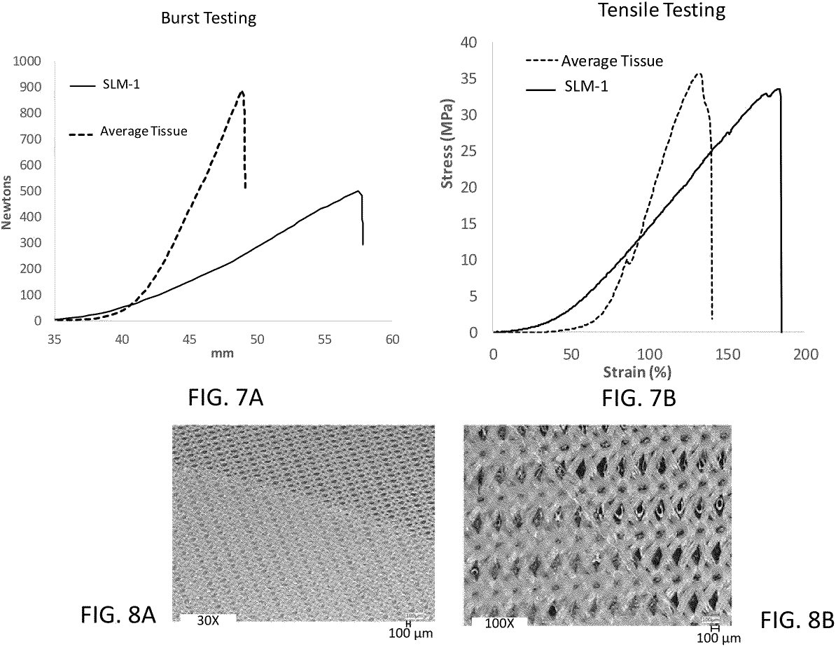

A knitted PET cloth (FIG. 6) was prepared with an 18-filament PET flat-drawn warp knit quality yarn (33 dtex/18 filaments). The cloth was warp knitted and scoured, with construction of 40±5 Wales/inch (16±2 Wales/cm), 90±10 course/inch (35±4 course/cm). The burst strength (based on ASTM D3887-96 Standard Specification for Tolerances for Knitted Fabrics, and ASTM D3787-01 Standard Test Method for Bursting Strength of Textiles - Constant Rate of Traverser (CRT) Ball Burst Test) was determined to be 356 N (80 lbf). In comparison, the burst strength of pericardium tissue over 30 samples varied from 450-700 N (100-160 lbf) (FIG. 7A). The tensile strengths of the PET cloth and pericardium tissue were similar (FIG. 7B). The PET cloth surface was hydrolyzed as described above, and the resulting reinforcement layer was dried in an oven at 45° C. overnight.

SLMs were made by attaching one or two layers of a PCU protective membrane to the dried reinforcement layer to form the intermediate layer. The PCU membranes were aromatic and aliphatic thermoplastic polyurethane (TPU) films having a thickness within a range of 25-127 µm (0.001″-0.005”), and Shore durometers of 75 A. Heat pressing was performed at 380° F. (190° C.) at 100-120 psi (0.7-0.8 N/mm²) for 15 seconds to attach the intermediate layer to the reinforcement layer. Alternatively co-extrusion process was used to encapsulate the PET textile backbone at 175-215° C. (350-420° F.) using a die extruder and then a molding press to control the final thickness of the film.

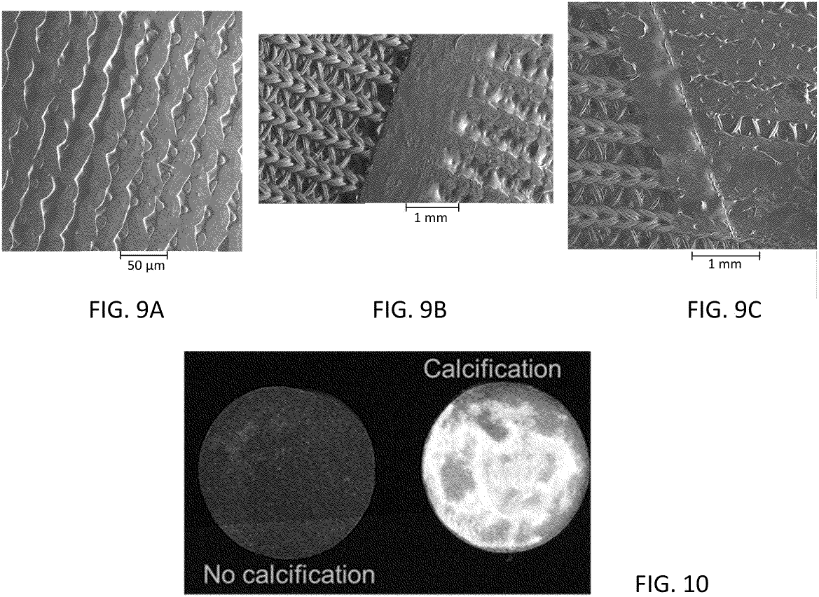

FIGS. 8A and 8B are microscope images of the dried reinforcement layer and the SLM comprising the PET/hydrolyzed PET reinforcement layer and the TPU protection membrane. FIGS. 9A-9C are scanning electron microscope (SEM) images of the full coverage of the reinforcement layer with TPU film (9A, 103X), uncoated reinforcement layer (left half of 9B), and partial coverage of the reinforcement layer with defects in the TPU film (right half of 9C). The SLMs were evaluated for burst strength (ASTM D3887-96, ASTM D3787-01). The results are shown in Table 1.

TABLE 1

| | | | |
|---|---|---|---|
| TPU thickness | SLM thickness | Burst force | SEM analysis |
| 25 microns | 0.21 mm | 415-430 N | 1-layer partial encapsulation 2-layer complete encapsulation |
| 76 microns | 0.21 mm | 430-460 N | 1-layer complete encapsulation 2-layer complete encapsulation |
| 127 microns | 0.38 mm | 470-490 N | 1-layer complete encapsulation 2-layer complete encapsulation |

Longer term mechanical performance of valves : Synthetic leaflet material was manufactured as described above and accelerated wear testing was performed with more than 200 million cycles or 300 million cycles, with passing results per ISO 5840-1.

SLM material in vivo calcification in a rabbit intramuscular model: SLM samples were implanted intramuscularly into rabbits according to the previously published methodology (Wright et al., Comp Med. 2009, 59(3):266). The intramuscular rabbit model has proven to be a fast and aggressive differentiator of anti-calcification treatment. Each rabbit received one disc from each sample group and the position of the discs was randomized.

All rabbits need to survive the implantation and monitoring duration. The discs are retrieved at 60 days after implantation. The discs from two rabbits are explanted with surrounding muscle for histological evaluation. The remainder of the discs are subjected to calcium analysis by X-ray imaging as shown in FIG. 10 and quantified by elemental analysis using ICP-OES.

Valves in vivo calcification resistance assessment: An adolescent/juvenile sheep model is sensitive in the study of calcification process on heart valve prostheses, as reported in The Journal of Thoracic and Cardiovascular Surgery 2006, (132) 1:89-98. Valves in mitral and aortic positions are implanted in sheep, younger than 12 months of age and weighing between 29 and 63 kg, for 3-6 moths to assess valve calcification. After explantation, calcium presence on the leaflets is determined by X-ray imaging. Then cross-sections of the leaflets are sent out for histological evaluation. The remainder of the leaflets are subjected to calcium quantification by elemental analysis using ICP-OES.

### Example 3

### Pet/Polyether-Based Hydrogel Polyurethane Synthetic Leaflet Material

SLMs comprising a polyether-based hydrogel thermoplastic polyurethane (HTPRU) protection membrane were prepared and characterized. Films were applied to the reinforcement layer of Example 2 at a temperature of 385° F. (196° C.) at 90 psi (0.6 N/m²). The film properties are shown in Table 2, where TPU thickness, SLM strength, and melting temperature were measured on the dry material.

TABLE 2

| TPU thickness | Durometer | SLM strength | Melting temp. | Water absorption | SEM Analysis |
|---|---|---|---|---|---|
| 76 µm | 85 A | 40-50 MPa | 193° C. | 30-35% | 1-layer complete encapsulation |
| 76 µm | 85 A | 50-60 MPa | 193° C. | 15-20% | 1-layer complete encapsulation |
| 76 µm | 100 A | 10-15 MPa | 188° C. | 50-60% | 1-layer complete encapsulation |
| 76 µm | 85 A | 50-60 MPa | 193° C. | 50-60% | 1-layer complete encapsulation |

### Example 4

### Synthetic Leaflet Materials With Zwitterionic Polymer Coating

SLMs may be coated with a zwitterionic polymer to enhance surface chemistry. A PCU-coated SLM as described in Example 2 was coated with a polymer comprising 2-methacryloyloxyethyl phosphorylcholine (MPC). The zwitterionic phosphorylcholine side chain exhibits excellent resistance to nonspecific protein adsorption, cell adhesion, and/or blood coagulation. Two MPC polymers were evaluated - Lipidure® CM5206 and Lipidure® AC 01 (NOF America, White Plains, NY). In Lipidure® CM5206, R′ is a hydrophobic group, an anionic group, a cationic group, a hydrogen-bonding group, a photoreactive group, or an alkoxy silane group; and m and n are integers. In Lipidure® AC 01, m and n are integers.

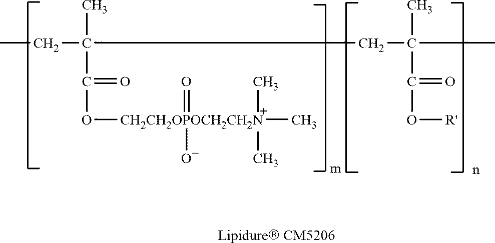

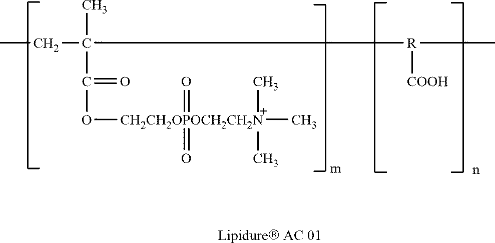

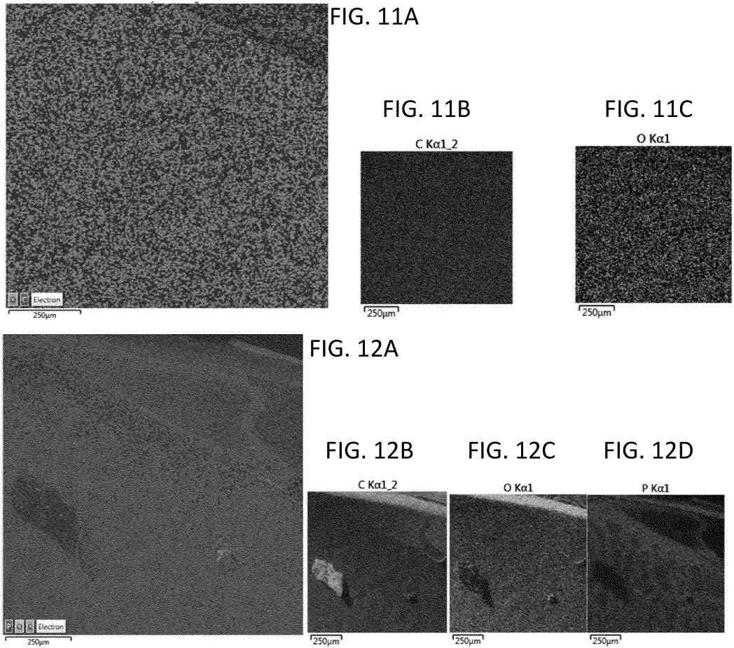

An MPC solution comprising 2-3 wt% Lipidure® CM5206 in ethanol was prepared (e.g., 0.257 g in 10 mL). The MPC was mechanically attached to the SLM by dipping the SLM into the MPC solution for one minute. Ethanol swelled the exposed surface of the TPU protection film, allowing the MPC molecules to insert between TPU polymer chains, providing a mechanical attachment as the ethanol evaporates and the TPU swelling disappears. The MPC-coated SLM was dried room temperature or at 50° C. for one hour. FTIR-ATR testing was performed to confirm the presence of the MPC coating on the SLM. Energy-dispersive X-ray spectroscopy (EDS)/SEM analysis was performed to confirm MPC coverage of the SLM. FIGS. 11A-11C are layered (11A), carbon (11B), and oxygen (11C) images of the SLM prior to MPC coating. FIGS. 12A-12D are layered (12A), carbon (12B), oxygen (12C), and phosphorus (12D) images of the MPC-coated SLM.

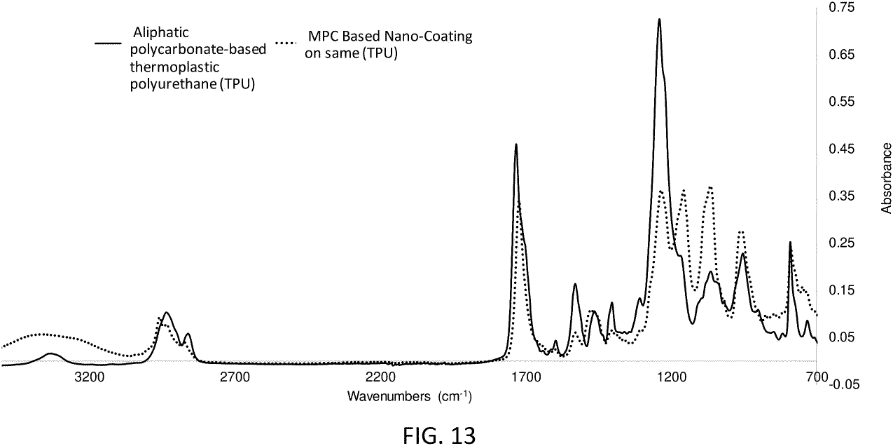

Surface analysis using FTIR-ATR was used to confirm the MPC coating and the surface chemistry. Transmittance absorption peaks at 1240, 1080, and 970 cm⁻¹ were observed only for TPU with an MPC coating. The absorption near 1725 cm⁻¹ observed for TPU-MPC corresponds to the carbonyl in the MPC unit. FIG. 13 shows spectra of the uncoated SLM and MPC-coated SLM. Bonded and non-bonded urethane bands presented at 1704 cm⁻¹ and 1728 cm⁻¹, a relatively weaker band at 1639 cm⁻¹ due to amide I, and a strong band at 963 cm⁻¹ due to trans-1,4 addition of HC=CH were present. Absorption bands for phosphocholine (PO₄, N—H, C═O bonds), and quaternary ammonium were also observed. The MPC coating is confirmed by the presence of the additional 1156 cm⁻¹ peak.

Another MPC solution comprising 5 wt% Lipidure® AC 01 in water is prepared and mixed with 10 mL of ethanol, and used to treat an SLM as detailed above for Lipidure® CM5206. Briefly, the SLM was treated with dilute acetic acid or plasma to produce additional carboxylic acid groups on the TPU protection membrane. The MPC was chemically attached to the SLM by dipping the SLM into the MPC solution for one minute. The MPC-coated SLM was dried at room temperature or at 50° C. for one hour.

The MPC-coated SLM was characterized by tensile testing (ASTM412) and stress relaxation (ASTM D6048). Stress relaxation was measured by subjecting the sample to a 1-MPa load. Oxidative biostability and water absorption over time are important measures of SLM performance. The SLM material was aged in saline solution at 60° C. for a month after exposure to 30% H₂O₂ at 50° C. The SLM leaflets were removed from the aging solution, and dimensional analysis of the leaflet was performed using a high-accuracy digital microscope. Ball burst testing (ASTM3787) was performed. Creep/fatigue testing were performed by dynamic mechanical analysis. The T_{g} of the PET reinforcement layer was assessed by differential scanning calorimetry from 30° C. to 200° C. at 5° C./min using a DSC 4000 system from Perkin Elmer (Waltham, MA).

Longer term mechanical performance assessment: Synthetic leaflet material was manufactured as described above and accelerated wear testing (AWT) was performed with more than +300 million cycles with passing results per ISO 5840-1.

In rabbit calcification assessment via intramuscular implantation for 90 days showed no Ca+ presence by X-ray nor inductive plasma mass spectroscopy (ICP-MS) analyses.

Calcification assessment in juvenile sheep (3-6 months) in valves sizes 21-25 \-mm showed no calcification as assessed by X-ray nor inductive plasma mass spectroscopy (ICP-MS) analyses.

### Example 5

### PCU-PGS Synthetic Leaflet Material

A reinforcement layer was formed with aromatic polycarbonate polyurethane (PCU) (Carbothane™ AC-4075A, Lubrizol Advanced Materials, Inc., Cleveland, Ohio) filaments using an electrospinning process. An intermediate layer comprising a poly(glycerol sebacate) (PGS, weight-average molecular weight 300,000 g/mol) and a thermoplastic polyurethane (TPU) protection membrane were chemically attached to the surfaces of the reinforcement layer. For the fabrication of TPU-PGS films (TPU such as Pellethane 80A or Carbothane ₇₅A), pellets were dissolved in one or a mixture of organic solvents (chloroform (CF) /N,N-dimethylformamide (DMF) (v/v=6:4), tetrahydrofuran (THF), DMAc, acetone 1,1,1,3,3,3-hexafluoroisopropanol (HFIP), or a binary solvent of 2,2,2-trifluoroethanol (TFE) and acetic acid) to achieve a 3-10% (w/v) solution. The PGS pellets were also dissolved in CF, DMF, DMAC and/or acetone. Both solutions then were combined to provide TPU/PGS at various polymer ratios (6:6, 6:4, and 6:2). The concentration of PCU was kept at 3-10% (w/v).

Morphological characterization using scanning electron microscopy(SEM) and Fourier Transform Infrared Spectrometry (FTIR-ATR) was used to determine surface chemistry and morphology to assure film homogeneity. The fiber diameters and diameter distributions were analyzed from SEM images. At least fifty fibers were measured from three SEM images to calculate the average geometric information. The mean pore size and pore size distributions of electrospun PCU scaffolds were determined.

In rabbit calcification assessment via intramuscular implantation for 90 days showed no Ca+ presence by X-ray nor inductive plasma mass spectroscopy (ICP-MS) analyses.

### Example 6

### Polyurethane-PEG-Mpc Synthetic Leaflet Material

A reinforcement layer was formed by simultaneous electrospinning of two polyurethanes comprising an aliphatic, hydrophilic polyether-based polyurethane (Tecophilic™ HP-60D-20, Lubrizol Advanced Materials, Inc., Cleveland, Ohio) hydrogel and a biostable aromatic polycarbonate polyurethane (PCU) (Carbothane™ 4075A, Lubrizol Advanced Materials, Inc.). An intermediate layer comprising a PEG-based hydrogel protection membrane was formed by dip-coating the reinforcement layer in a prepolymer solution containing PEGDA (poly(ethylene glycol) diacrylate, 10 kDa, 10 wt%) and 0.5 wt% 2-hydroxy-4′-(2-hydroxyethoxy)-2-methylpropiophenone (photoinitiator) in phosphate buffered saline (PBS, pH = 7.4), and subsequently polymerizing for 9 minutes under ultraviolet light (centered at 367 nm, 6 mW/cm²). The implantable material was dip-coated in a solution comprising the zwitterionic polymer poly(2-methacryloyloxyethyl phosphorylcholine) (Lipidure® PC, NOF America, White Plains, NY) dissolved in ethanol at 2% v/v, thereby forming an outer layer comprising the zwitterionic polymer on the surface.

### Example 7

### Pet-Poly(HEMA)-Mpc Synthetic Leaflet Material

A reinforcement layer was formed by warp knitted PET cloth as in Example 2. An intermediate layer comprising poly(2-hydroxyethyl methacrylate) (poly(HEMA)) (MW 300,000-1,000,000) was dissolved in DMF and applied to the PET cloth by spray coating on the reinforcement layer. The implantable material was dip-coated in a solution comprising the zwitterionic polymer poly(2-methacryloyloxyethyl phosphorylcholine) (Lipidure® PC, NOF America, White Plains, NY) dissolved in ethanol at 2% v/v, thereby forming an outer layer comprising the zwitterionic polymer on the surface.

### Example 8

### Silk-Polyether Polyurethane Synthetic Leaflet Material

A reinforcement layer was made from warp or weft knitted pure silk and then encapsulated into aromatic polycarbonate polyurethane or aliphatic polyether polyurethane by a compression molding process. A cloth was warp knitted and scoured, with construction of 40±5 Wales/inch (16±2 Wales/cm), 90±10 course/inch (35±4 course/cm). The burst strength (based on ASTM D3887-96 Standard Specification for Tolerances for Knitted Fabrics, and ASTM D3787-01 Standard Test Method for Bursting Strength of Textiles -Constant Rate of Traverser (CRT) Ball Burst Test) was determined to be 356 N (80 lbf). In comparison, the burst strength of pericardium tissue over 30 samples varied from 450-700 N (100-160 lbf) (FIG. 7A). The tensile strength of the silk-based SLM was higher than pericardium tissue. Biostability assessment of the final film was performed under oxidative aging, suggesting silk will maintain its strength for at least 2-3 years of implantation.

### Example 9

### Upy Polymer-Silk-Polyester Synthetic Leaflet Material

UPy (ureidopyrimidinone) technology polymer (antifouling coating) was synthesized from 2-amino-5-(2-hydroxyethyl)-6-methyl-4(3H)-pyrimidinone, hexane diisocyanate, hexanediol and hydrogenated polybutadiene diol (Mn = 2000), using Class 3 solvents. After polymer manufacturing, residual solvents were lower than 5000 ppm. The obtained polymer had a low modulus between 0.6-10 MPa. GPC in THF vs Pst-standards: Mn = 44 kDa, Mw = 72 kDa. Polymer identity confirmed with FT-IR. Mechanical testing as shown in previous presentation (dog-bone at 50 mm/min) Films were cast from 8-10 w% solutions in THF, dried for at least 14 days under atmospheric conditions (after 2 days removed from mold), no vacuo has been used.

UPy polymer was incorporated into a silk and polyester (PET) warp and weft knit cloth by solvent casting from THF solution followed by atmospheric drying. The obtained material thickness varied between 0.2-0.6 mm.

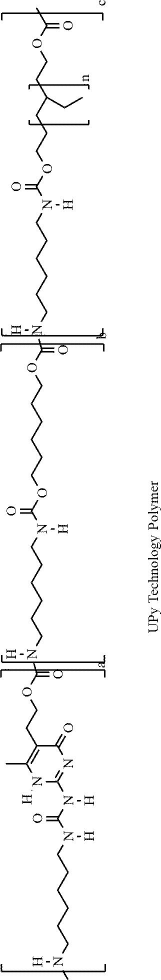

In rabbit calcification assessment via intramuscular implantation for 90 days showed no Ca+ presence by X-ray imaging nor inductive plasma mass spectroscopy (ICP-MS) analyses.

### Example 10

### Gelatin-Polycarbonate Polyether Polyurethane Synthetic Leaflet Material

A reinforcement layer made from electrospun gelatin and further crosslinked by EDC/NHS and/or genipin cross-linked gelatin was then dip coated from THF solution with polycarbonate and polyether polyurethanes (Carbothane AC and PC 75A-100A) to achieve a final thickness between 0.2 to 0.6 mm. ASTM D3787-01 Standard Test Method for Bursting Strength was performed revealing acceptable stiffness and UTS values.

### Example 11

### Polycarbonate Polyether Polyurethane-PGS-PGSU Synthetic Leaflet Material

A reinforcement layer was made from electrospun polycarbonate and polyether polyurethanes (Carbothane AC and PC 75A) at high and low density with pore sizes ranging between 0.1 and 45 microns. PGS and PGSU (TPU with PGS) polymer was used as an antifouling coating and was applied by spray coating from toluene until a thickness range between 0.2-0.6 mm was reached.

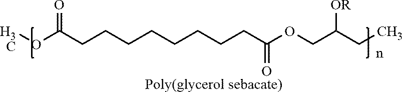

The electrospinning parameters were as follows: applied voltage was 18 kV, the flow rate was kept at 0.5 mL/h, and the distance from the needle to the collector was 20 cm. The temperature and humidity at ambient condition were around 25° C. and 30%, respectively. Flat aluminum foil was used as a collector to gather all of the electrospun fibers.

In rabbit calcification assessment via intramuscular implantation for 90 days showed no Ca+ presence by X-ray nor inductive plasma mass spectroscopy (ICP-MS) analyses.

In view of the many possible examples to which the principles of the disclosure may be applied, it should be recognized that the illustrated examples are only preferred examples and should not be taken as limiting the scope thereof. Rather, the scope is defined by the following claims. We therefore claim all that comes within the scope and spirit of these claims.

What is claimed is:

1. An anti-fouling implantable material, comprising:
a reinforcement layer comprising a plurality of polymeric filaments comprising a filament polymer, the reinforcement layer having a first surface and an opposing surface;
an intermediate layer comprising a protection membrane attached to at least a portion of the first surface, the protection membrane comprising a protective polymer, optionally wherein the intermediate layer further comprises a second protection membrane attached to at least a portion of the opposing surface of the reinforcement layer, the second protection membrane comprising a second protective polymer, wherein the second protective polymer of the second protection membrane may have the same or a different chemical composition than the protective polymer; and
an outer layer comprising an ionic polymer grafted onto an exposed surface of the intermediate layer.

2. The anti-fouling implantable material of claim 1, wherein the polymeric filaments of the reinforcement layer:
are randomly oriented to form a material comprising entangled polymer filaments; or
are aligned unidirectionally; or
form an interwoven mesh comprising a first plurality of polymeric filaments having a first common extending direction interwoven with a second plurality of polymeric filaments having a second common extending direction, the second common extending direction orthogonal to the first common extending direction; or
form an intra-lamellar mesh comprising a plurality of lamellae, wherein polymeric filaments in each lamella have a common extending direction, and polymeric filaments in adjacent lamellae are oriented in different extending directions; or
are knitted to form a knitted material; or
are twisted into yarn fibers comprising a plurality of polymeric filaments, wherein the yarn fibers subsequently are (i) randomly oriented to form a material comprising randomly oriented, entangled yarn fibers, (ii) aligned unidirectionally, (iii) woven to form an interwoven mesh, (iv) aligned to form an intra-lamellar mesh comprising a plurality of lamellae, or (v) knitted to form a knitted material.

3. The anti-fouling implantable material of claim 1, wherein the filament polymer comprises a polyurethane, a polyether ketone, a poly(ethylene terephthalate), a polycarbonate, a polyester, a polyacrylate, a polysiloxane, an aromatic polyolefin, an aliphatic polyolefin, a polyamide, a glycerol-ester polymer, a polycarboxylic acid, a polysulfone, a polysaccharide, a polyamine, a polyamino acid, a polypeptide, or any combination thereof.

4. The anti-fouling implantable material of claim 1, wherein:
the filament polymer is a biostable polymer comprising a polyurethane, a polyester, poly(ethylene terephthalate), a polycarbonate, a polysiloxane, an aromatic polyolefin, or an aliphatic polyolefin; or
the filament polymer is a biodegradable polymer comprising poly(lactic acid), poly(lactic-co-glycolic acid), a polysaccharide, a polyamino acid, a polypeptide, or poly(glycerol sebacate).

5. The anti-fouling implantable material of claim 1, wherein the polymeric filaments comprise a core and a shell surrounding the core, wherein the core comprises the filament polymer and the shell comprises a shell polymer.

6. The anti-fouling implantable material of claim 5, wherein:
the shell polymer is a biodegradable polymer comprising poly(lactic acid), poly(lactic-co-glycolic acid), a polysaccharide, a polyamino acid, a polypeptide, or poly(glycerol sebacate); or
the shell polymer is a biostable polymer comprising hydrolyzed poly(ethylene terephthalate) or a polyurethane.

7. The anti-fouling implantable material of claim 1, wherein the polymeric filaments have an average diameter of from 0.001 µm to 2000 µm.

8. The anti-fouling implantable material of claim 1, wherein the reinforcement layer has:
(i) a thickness of from 25 µm to 500 µm; or
(ii) a burst strength of from 50-800 N; or
(iii) a tensile strength of from 50-800 N; or
(iv) any combination of (i), (ii), and (iii).

9. The anti-fouling implantable material of claim 1, wherein the protective polymer comprises:
a biostable synthetic polymer selected from polyethylene, polypropylene, polytetrafluoroethylene, a polyether, polycarbonate polyurethane, polysiloxane polyurethane, a polyether polyurethane elastomer, a polyester polyurethane elastomer, a silicone, a polycarbonate, a polysulfone, polyether ether ketone, poly(ethylene terephthalate), a polyester, or any combination thereof; or
a biodegradable synthetic polymer selected from a polyester, a polyacrylate, a polyamide, a hydrophilic polyester polyurethane, a hydrophilic polyurea, poly(amide-enamine), a polyanhydride, a poly(ester amide), poly(glycolide), polylactic acid, polyglycolic acid, polycaprolactone, poly(hydroxy butyrate), poly(ε-caprolactone), poly(vinyl alcohol)-hyaluronic acid, a hyaluronate amine, a ureidopyrimidinone-based polymer, or any combination thereof; or
a hydrogel-forming polymer selected from a protein, a polysaccharide, a hydrophilic polyurethane, poly(ethylene oxide), polyacrylamide, a polyethylene glycol, a polyacrylate, a polypeptide, poly(glycerol sebacate), poly(xylitol sebacate), or any combination thereof.

10. The anti-fouling implantable material of claim 1, wherein the intermediate layer has:
(i) an average thickness of from 0.1 µm to 100 µm; or
(ii) a durometer Shore hardness of from 10 A to 80 A; or
(iii) a flexural modulus of from 1 N/mm² to 50 N/mm²; or
(iv) a dry ultimate tensile strength of from 10 N/mm² to 60 N/mm²; or
(v) a wet ultimate tensile strength of from 5 N/mm² to 40 N/mm²; or
(vi) any combination of (i), (ii), (iii), (iv), and (v).

11. The anti-fouling implantable material of claim 1, wherein the ionic polymer is an anionic polymer, a cationic polymer, or a zwitterionic polymer.

12. The anti-fouling implantable material of claim 11, wherein the ionic polymer comprises a poly(phosphocholine), a poly(sulfobetaine), a poly(carboxybetaine), a zwitterionic polysaccharide, diethyl ethanolamine quaternized with 2-acrylamide-2-methylpropane sulfonic acid and acrylic acid, or any combination thereof.

13. The anti-fouling implantable material of claim 11, wherein the ionic polymer comprises:
poly(2-methacryloyloxyethyl phosphorylcholine) (MPC); or
poly(MPC-co-2-ethylhexyl methacrylate-co-N,N-diethylaminoethyl methacrylate); or
poly(MPC-co-p-nitrophenyloxycarbonyl poly(ethylene glycol) methacrylate); or
a poly(₂-hydroxyethyl methacrylate)-MPC copolymer; or
a polyvinylpyrrolidone-MPC copolymer; or
any combination thereof.

14. The anti-fouling implantable material of claim 1, wherein the outer layer has an average thickness of from 0.001 µm to 25 µm.

15. An implantable medical device comprising an anti-fouling implantable material according to claim 1.

16. The implantable medical device of claim 15, wherein the implantable medical device comprises a prosthetic heart valve, a vascular graft, an annuloplasty ring, a cardiovascular patch, or a coaptation clip.

17. The implantable medical device of claim 15, wherein the implantable medical device comprises a prosthetic heart valve comprising a plurality of leaflets, a sealing skirt, a covering for a metal component, or any combination thereof, formed from the anti-fouling implantable material.

18. A method for making an anti-fouling implantable material, comprising:
forming an intermediate layer comprising a protection membrane on at least a portion of a first surface of a reinforcement layer, the reinforcement layer comprising a plurality of polymeric filaments comprising a filament polymer, and the protection membrane comprising a protective polymer; and
forming an outer layer by grafting an ionic polymer onto an exposed surface of the intermediate layer.

19. The method of claim 18, further comprising forming the reinforcement layer by:
jet spinning, electro-spinning, or melt spinning the plurality of polymeric filaments to form a material comprising randomly oriented, entangled filaments; or
aligning the plurality of polymeric filaments unidirectionally; or
weaving the plurality of polymeric filaments to form an interwoven mesh comprising a first plurality of filaments having a first common extending direction interwoven with a second plurality of filaments having a second common extending direction, the second common extending direction orthogonal to the first common extending direction; or
aligning the plurality of polymeric filaments to form an intra-lamellar mesh comprising a plurality of lamellae, wherein filaments in each lamella have a common extending direction, and filaments in adjacent lamellae are oriented in different extending directions; or
knitting the plurality of polymeric filaments to form a knitted material; or
twisting the plurality of polymeric filaments to form yarn fibers, and subsequently (i) randomly orienting the yarn fibers for form a material comprising randomly oriented, entangled yarn fibers, (ii) aligning the yarn fibers unidirectionally, (iii) weaving the yarn fibers to form an interwoven mesh, (iv) aligning the yarn fibers to form an intra-lamellar mesh comprising a plurality of lamellae, or (v) knitting the yarn fibers to form a knitted material; or
printing the plurality of polymeric filaments in a pattern by three-dimensional printing.

20. The method of claim 18, further comprising forming the plurality of polymeric filaments comprising the filament polymer by jet spinning, electro-spinning, melt spinning, three-dimensional printing, extrusion, or a melt-blown process.

21. The method of claim 18, wherein the polymeric filaments comprise a core comprising the filament polymer and a shell surrounding the core, the shell comprising a shell polymer, the method further comprising:
forming the core and shell in a single step by jet spinning, electro-spinning, coextrusion or three-dimensional printing; or
forming the core and then coating the core with the shell polymer to form the shell; or
forming the core, and hydrolyzing a surface of the core to form a shell.

22. The method of claim 18, wherein forming the intermediate layer comprising the protection membrane on at least a portion of the first surface of the reinforcement layer further comprises:
thermally attaching the protection membrane to at least a portion of the first surface; or
mechanically attaching the protection membrane to at least a portion of the first surface; or
ultrasonically attaching the protection membrane to at least a portion of the first surface; or
attaching the protection membrane to at least a portion of the first surface using a laser; or
chemically attaching the protection membrane to at least a portion of the first surface by hydrolysis or oxidation of the reinforcement layer and protective polymer, whereby chemical functional groups of the filament polymer or shell polymer react with functional groups of the protective polymer; or
coating the reinforcement layer with a solution comprising the protective polymer and a solvent, and removing the solvent to form the protection membrane; or
forming the protection membrane from a solution comprising the protective polymer by a reactive dip-coating process; or
coating the reinforcement layer with a solution comprising the protective polymer and curing the protective polymer by ultraviolet irradiation; or
printing the protection membrane onto at least a portion of the first surface by a three-dimensional printing process.

23. The method of claim 18, wherein grafting the ionic polymer onto the exposed surface of the intermediate layer comprises:
coating the exposed surface with a solution comprising the ionic polymer to form an ionic polymer-coated material; and
drying the ionic polymer-coated material.

24. The method of claim 18, wherein the ionic polymer comprises a poly(phosphocholine), a poly(sulfobetaine), a poly(carboxybetaine), a zwitterionic polysaccharide, diethyl ethanolamine quaternized with 2-acrylamide-2-methylpropane sulfonic acid and acrylic acid, or any combination thereof.

25. The method of any of claim 18, further comprising forming a plurality of leaflets from the anti-fouling implantable material and coupling the leaflets to a frame of a prosthetic heart valve.
